(19) United States
(12) Patent Application Publication
(10) Pub. No.: US 2011/0033897 A1
(43) Pub. Date: Feb. 10, 2011

(54) Method for the Obtainment of Hydrolytic Enzymes, Hydrolytic Method for Producing Fermentable Sugars, Additives comprising Fermentable Sugars, and Process for Producing Ethanol from Sugar Cane Bagasse

(76) Inventors: Pinheiro Dillon Aldo Jose, Rio de Janeiro (BR); Alexandra Amorim Salgueiro, Recife (BR); Carlos Fernandes Das Chagas, Caruaru (BR); Jose Augusto Travassos Rios Tome, Recife (BR); Henrique Macedo Baudel, Recife (BR); Marli Camassola, Caxias do Sul (BR)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/528,469

(22) PCT Filed: Mar. 3, 2008
(86) PCT No.: PCT/BR08/00058

§ 371 Date: Oct. 29, 2010

(30) Foreign Application Priority Data
Mar. 2, 2007 (BR) PI 0700481-8
Mar. 2, 2007 (BR) PI 0702162-3
Jul. 6, 2007 (BR) PI 0703302-8

(51) Int. Cl. C12P 19/00; C12P 7/10; C12N 9/42; C12N 9/24; C08B 37/00; C12N 1/00

(52) U.S. Cl. 435/72; 435/165; 435/209; 435/200; 127/36; 435/243

(57) ABSTRACT

The present invention provides industrial methods involving the hydrolysis of sugar cane bagasse, including methods for producing hydrolytic enzymes, for hydrolysis of sugar cane bagasse, for the production of additives comprising fermentable sugars and the respective additives, and methods of producing ethanol. The invention methods comprised the submerged culture of Penicillium echinulatum strain 9A02S1 (DSM18942), using sugar cane bagasse in natura and/or pretreated. The additives of the invention are obtained from treating the sugar cane bagasse with enzymes resulting from the culture of said Penicillium echinulatum, being useful, among other things, in the industrial production of ethanol.

### BACKGROUND OF THE INVENTION

1. Technical Field

The present invention refers to industrial methods involving the hydrolysis of sugar cane bagasse. More specifically, the present invention comprises industrial methods for the production of hydrolytic enzymes, for the enzymatic hydrolysis of sugar cane bagasse, for the production of additives comprising fermentable sugars and to the respective additives, and methods of producing ethanol. The invention methods comprised the submerged culture of Penicillium echinulatum strain 9A02S1 (DSM18942), using sugar cane bagasse in natura and/or pretreated as substrate. The additives of the invention are obtained from treating the sugar cane bagasse with enzymes resulting from the culture of said Penicillium echinulatum, being useful, among other things, in the industrial production of ethanol.

2. Related Art

The production of fermentable sugars from vegetable biomass in general involves the use of cellulase and/or xylanase enzymes, and pulps containing cellulose and hemicellulose as substrate are commonly used. Cellulose pulp is a commercial product of the paper industry, which costs around US$130/ton. The high cost of cellulosic pulp is one of the facts that contribute to the high price of enzymatic mixtures (cellulases and hemicellulases), the production yield of which generally does not exceed 300 Ul/g of cellulose. This cost is determinant when considering the production of cellulases and hemicellulases for any industrial applications of these enzymes, particularly the hydrolysis of lignocellulosic materials, with a view to producing fermentable sugars and/or ethanol. For the production method of this fuel made from lignocellulosic biomasses to be economically feasible, calculations suggest a maximum enzymes cost of US$0.18 per liter of ethanol produced.

A cheap source of cellulose and with a high potential for the production of fermentable sugars and/or ethanol is sugar cane bagasse,—a subproduct of the production of sugar and ethanol, provided that a method is available in which the enzymatic stages (production of enzymes and enzymatic hydrolysis) are technically and economically viable. The present invention aims to meet these demands.

The structure of lignocellulosic biomass present in sugar cane bagasse constitutes a barrier for enzyme access to the cellulosic matrix, so there is a need to pretreat this lignocellulosic residue. The pretreatment of lignocellulosic biomass is required to promote physical-chemical alterations in its structure, allowing the cellulosic matrix to become more accessible to the enzymes that convert carbohydrate polymers into fermentable sugars. Specifically, during pretreatment, the casing of lignin and hemicelluloses is broken and the crystalline structure of the cellulose is altered, making the cellulosic matrix is much more susceptible to enzymatic attack. Physical and/or chemical pretreatment methods consist of fragmenting the lignocellulosic biomass into smaller structures, increasing their accessibility to hydrolytic agents.

Patent literature comprises various documents on the production of cellulases and hemicellulases, as well as methods of using vegetable cellulosic biomasses for producing fermentable sugars and/or ethanol. International patent application WO 06/113683, entitled “Process for the production of animal feed and ethanol and novel animal feed”, describes a method of processing corn seeds, where the part rich in proteins is used to produce animal fodder, while the amylasic and lignocellulosic portions are hydrolyzed with cellulase, hemicellulase and amylases. The monosaccharides obtained from this hydrolysis are used in the production of ethanol.

North American patent U.S. Pat. No. 6,569,646, entitled “Process for the production of an enzyme preparation containing xylanase and carboxymethyl cellulase from Termitomyces clypeatus”, discloses a method of producing carboxymethylcellulases and xylanases from Termitomyces clypeatus—MTCC 5091. This microorganism is grown in a medium containing sources of carbon, nitrogen and micronutrients in a pH from 3 to 7 and an incubation temperature of 25-30° C. The sources of carbon used are agricultural residues—amide free from wheat bran and corn, both in powder form, while the sources of nitrogen are ammonium chloride, ammonium nitrate, ammonium phosphate, bacteriological peptone, soy peptone, malt extract, wheat bran and milhocina.

North American patent U.S. Pat. No. 4,966,850, entitled “Production of thermostable xylanase and cellulase”, discloses the production of thermostable cellulotic and xylanolytic enzymes, particularly xylanase and cellulase, by the microorganism Thermoascus aurantiacus in a culture medium containing cellulose or hemicellulose as substrate.

There are also other known patents relating to methods for using to vegetable cellulosic biomasses for producing enzymes and/or fermentable sugars. Table 1 shows some patents and the respective microorganisms used.

TABLE 1
Patents and microorganisms used to produce enzymes and/or
fermentable sugars.

| Pat. No. | Microorganism |
|---|---|
| U.S. Pat. No. 6,566,112 | Actinomycete |
| U.S. Pat. No. 6,451,063 | Thermomonospora fusca |
| U.S. Pat. No. 6,428,996 | Piromyces rhizinflata |
| U.S. Pat. No. 6,287,839 | Actinomycete |
| U.S. Pat. No. 6,114,296 | Humicola insolens |
| U.S. Pat. No. 6,063,611 | Bacillus sp. CBS 669.93 |
| U.S. Pat. No. 6,277,596 | Trichoderma viride |
| U.S. Pat. No. 5,861,271 | Trichoderma longibrachiatum |
| EP 20060113064 | Humicola |
| EP 1627050 | Humicola grisea CBH1.1 |
| EP 1618182 | Bacillus |
| WO 2004/016760 | Hyprocrea jecorina |

Although the documents cited mention the production of enzymes and/or fermentable sugars, the methods and products of the present invention are innovative, since none of the methods cited previously describes technology using the microorganism Penicillium echinulatum, which provides substantial technical and economic advantages compared with similar known methods. The microorganism used in the present invention is a filamentous fungus developed over the last 10 years at the University of Caxias do Sul by successive mutageneses and selections, having been identified as a good producer of cellulases and xylanases and deposited at the German resource center for biomaterial Deutsche Sammlung von Mikroorganismen und Zellkulturen (DSM) under number DSM18942, strain 9A02S1. The present invention presents various advantages in relation to the state of the art, such as:
production, on an industrial scale, of stable, high-activity enzymes, by submerged cultures using vegetable biomasses as substrates. Such enzymes are highly advantageous in hydrolytic process used on said vegetable biomasses;
production and obtention of additives comprising fermentable sugars, such additives being highly useful in the production of ethanol;
production of ethanol by using said fermentable sugars, substantially increasing the ethanol productivity yield compared to similar known processes.

The methods and products of the present invention provide attractive alternatives to the known processes, because they offer various technical and economic advantages, as described in greater detail ahead.

### BRIEF SUMMARY OF THE INVENTION

One of the objects of the present invention is to provide industrial methods to produce hydrolytic enzymes.

It is an object of the present invention to provide alternative methods of producing cellulases and/or hemicellulases.

In one aspect of the invention, thus being one of its objects, the methods for producing cellulases and/or hemicellulases of the invention comprise the culture of fungus Penicillium echinulatum strain 9A02S1 (DSM18942), both in submerged culture and in solid state fermentation.

It is another object of the present invention to provide an efficient and low-cost pretreatment process for sugar cane bagasse.

It is another object of the present invention to provide an efficient and low-cost culture medium for industrial bioprocesses.

In a preferred embodiment, the sugar cane bagasse is pretreated at temperatures in the range of 20 to 150° C., using sodium hydroxide in the concentrations of between 1 and 4% in relation to the lignocellulosic residue mass. Further, hydrogen peroxide and/or anthraquinone can be used in concentrations ranging from 0.1 to 2% and 0.001 to 2% of anthraquinone. Afterwards, the pH is adjusted to within the range of 4 and 7 with sulfuric acid. This biomass is hydrolyzed using quantities of enzymes in the range of 10 to 15 FPU, in addition to quantities over 5 U of endoglycanases, 3 U of β-glycosidases, 2 U of xylanases per gram of dry biomass. The fermentable sugars obtained are fermented, with a view to producing ethanol and/or other products.

It is another object of the present invention to provide industrial methods for producing fermentable sugars from sugar cane bagasse.

It is another object of the present invention to provide additives for industrial use comprising fermentable sugars. In a preferred aspect, thus being yet another object of the present invention, additives useful in the production of ethanol are provided.

It is yet another object of the present invention to provide industrial processes for producing ethanol.

These and other objects of the present invention will be better understood and valued after reading the detailed description of the invention.

### BRIEF DESCRIPTION OF THE DRAWING[[S]] FIGURES

Table 2 contains the demonstrations of the chromatographic figures relating to the experiments carried out in the present invention, indicating the numerical reference for the respective figures and the corresponding experimental conditions.

TABLE 2
List of figures (chromatograms) and respective experimental
conditions.

| FIG. | | Experiment | Time (h) | Characteristic |
|---|---|---|---|---|
| 1 | a | E1 | 0 | Sample 0 h, without enzyme |
| | b | | 24 | Hydrolyzed |
| | c | H1 | 12 | Hydrolyzed/Fermentate |
| | d | C1 | 12 | Sugar cane juice Fermentate |
| 2 | a | E2 | 0 | Sample 0 h, with enzyme |
| | b | | 5 | Hydrolyzed |
| | c | | 8 | Hydrolyzed |
| | d | | 21 | Hydrolyzed |
| | e | E2 Ferm | 12 | Fermentation, 12 hours, without dilution |
| 3 | a | E3 | 0 | Sample 0 h, with enzyme |
| | b | | 4 | Hydrolyzed |
| | c | | 8 | Hydrolyzed |
| | d | E3A | 21 | Hydrolyzed |
| | e | H3 Ferm | 12 | Fermentation, 12 hours, without dilution |
| 4 | a | H4 Ferm | 12 | Fermentation, 12 hours, without dilution |
| | b | C/H4 Ferm | 12 | Fermentate with 50% juice |
| | c | C/E4/E5 Ferm | 12 | Fermentate of sugar cane juice 10brix |
| 5 | a | H5 Ferm | 12 | Fermentate without dilution |
| | b | C/H5 Ferm | 12 | Fermentate with 50% juice |
| | c | Sugar cane juice | | Chromatographic profile/Diluted 1:10 |
| 6 | a | E11 HP | 9 | Hydrolyzed |
| | b | E11 HL | .33 | Depolymerization I |
| | c | E11 HF | 1 | Depolymerization II |
| | d | C/E11 pH 2 | 12 | Fermentation with 50% juice + E11HF |
| | | | | Diluted 1:2 with water |
| | e | E11 pH 2 | 12 | Fermentation E11HF diluted 1:2 with water |
| | f | C/E11 pH 3.5 | 12 | Fermentation E11HI + juice, |
| | | | | depolymerization III |
| | g | E11 pH 3.5 | 12 | Fermentation E11HI diluted 1:2 with |
| | | | | water, depolymerization III |
| 7 | a | E12 HP | 21 | Hydrolyzed |
| | b | E12 HL | 1 | Depolymerization I |
| | c | H12 HF | .33 | Depolymerization II |
| 8 | a | E13 HP | 9 | Hydrolyzed |
| | b | E13 HL | 1 | Depolymerization I |
| | c | E13 HF | .33 | Depolymerization II |
| 9 | a | E14 HP | 8 | Hydrolyzed |
| | b | E14 HL | 1 | Depolymerization I |
| | c | E14 HF | .33 | Depolymerization II |
| 10 | a | E15 HP | 12 | Hydrolyzed |
| | b | E15 HL | 1 | Depolymerization I |
| | c | E15 HF | .33 | Depolymerization II |
| 11 | a | E16 HP | 3 + 19 | with agitation + without agitation/ |
| | | | | Hydrolyzed |
| | b | E16 HL | 1 | Depolymerization I |
| | c | E16 HF | .33 | Depolymerization II |
| 12 | a | E16 HP | 9 | Hydrolyzed |
| | b | E16 HF | 1 | Depolymerization I |
| 13 | | E18 | 10 | Hydrolyzed |
| 14 | | E19 | 12 | Hydrolyzed |
| | | E19 H | .33 | Depolymerization I |
| | | E19 HPM | 1 | Depolymerization II |
| 15 | | E21 | 12 | Hydrolyzed |
| 16 | a | E22 H | 12 | Hydrolyzed |
| | | E22 HPM | 1 | Depolymerization II |
| 17 | | E23 | 9 | Hydrolyzed |
| | | E23 H | 9 | Depolymerization I |
| 18 | a | E30 | 6 | Hydrolyzed |
| | b | E30 DF | .33 | Depolymerization I |
| | c | E30 CTA | 12 | Fermentation with total consumption of |
| | | | | sugar M1, by the use of high cellular |
| | | | | concentration of Saccharomyces sp. |
| | d | H 30 Ferm | 6 | Fermentation without dilution |
| 19 | a | H31 | 11 | Hydrolyzed |
| | b | E31 DF | .33 | Depolymerization I |
| | c | E31 CTA | 12 | Fermentation with total consumption of |
| | | | | sugar M1, by the use of high cellular |
| | | | | concentration of Saccharomyces sp. |
| | d | H 31 Ferm | 8 | Fermentation without dilution |
| 20 | a | H 32 | 6 | Hydrolyzed |
| | b | E 32 DF | .33 | Depolymerization I |
| | c | E32 CTA | 12 | Fermentation with total consumption of |
| | | | | sugar M1, by the use of high cellular |
| | | | | concentration of Saccharomyces sp. |
| | d | H 32 Ferm | 12 | Fermentation without dilution |
| 21 | a | E 33 DF | 6 | Depolymerization I |
| | b | E33 CTA | 12 | Fermentation with total consumption of |
| | | | | sugar M1, by the use of high cellular |
| | | | | concentration of Saccharomyces sp. |
| | c | H33 Ferm | 8 | Fermentation without dilution |
| 22 | a | E34 DF | .33 | Depolymerization I |
| | b | E34 CTA | 12 | Fermentation with total consumption of |
| | | | | sugar M1, by the use of high cellular |
| | | | | concentration of Saccharomyces sp. |
| | c | H34 Ferm | 8 | Fermentation without dilution |
| 23 | a | E35 DF | | Depolymerization I |
| | b | E35 CTA | 12 | Fermentation with total consumption of |
| | | | | sugar M1, by the use of high cellular |
| | | | | concentration of Saccharomyces sp. |
| 24 | a | E37 DF | .33 | |
| | b | E37 CTA | 12 | Fermentation with total consumption of |
| | | | | sugar M1, by the use of high cellular |
| | | | | concentration of Saccharomyces sp. |
| | c | H37 Ferm | 8 | Fermentation without dilution |
| 25 | a | E38 DF | .33 | Depolymerization I |
| | b | E38 CTA | 12 | Fermentation with total consumption of |
| | | | | sugar M1, by the use of high cellular |
| | | | | concentration of Saccharomyces sp. |
| | c | H38 Ferm | 8 | Fermentation without dilution |
| 26 | a | E39 DF | .33 | Depolymerization I |
| | b | E39 CTA | 12 | Fermentation with total consumption of |
| | | | | sugar M1, by the use of high cellular |
| | | | | concentration of Saccharomyces sp. |
| | c | H39 Ferm | 8 | Fermentation without dilution |
| 27 | a | E 40 | 12 | Hydrolyzed |
| | b | E40 D | .33 | Depolymerization I |
| | c | H40 Ferm | 8 | Fermentation without dilution |
| | d | H40 2 Ferm | 12 | Fermentation with dilution 1:2, pH 3.5 |
| | e | H40 2 Ferm | 24 | |
| 28 | a | H41 | 12 | Hydrolyzed |
| | b | H41D | .33 | Depolymerization I |
| | c | H41 Ferm | 8 | Fermentation without dilution |
| | d | H41 Ferm | 12 | Fermentation without dilution |
| | e | H41 2 Ferm | 12 | Fermentation with dilution 1:2, pH 3.5 |
| | f | H41 2 Ferm | 24 | Fermentation with dilution 1:2, pH 3.5 |
| | g | H41 Ferm | 8 | Sugar cane juice added |
| 29 | a | H42 | 9 | Hydrolyzed |
| | b | H42 D | .33 | Depolymerization I |
| | c | H42 Ferm | 8 | Fermentation without dilution |
| | d | H42 Ferm | 12 | Fermentation without dilution |
| | e | H42 Ferm | 8 | Sugar cane juice added |
| 30 | a | H43 | 3 + 19 | 3 h with action + 19 h without action - |
| | | | | Hydrolyzed |
| | b | H43D | .33 | Depolymerization I |
| | c | H43 Ferm | 8 | Fermentation without dilution |
| | d | H43 Ferm | 12 | Fermentation without dilution |
| | e | H43 Ferm | 8 | Sugar cane juice added |
| 31 | a | H44 | 12 | Hydrolyzed |
| | b | H44D | .33 | Depolymerization I |
| | c | H44 Ferm | 8 | Fermentation without dilution |
| | d | H44 Ferm | 12 | Fermentation without dilution |
| | e | H44 2 Ferm | 12 | Fermentation with dilution 1:2, pH 3.5 |
| | f | H44 Ferm | 8 | Sugar cane juice added |
| 32 | a | H45 | 12 | Hydrolyzed |
| | b | H45D | .33 | Depolymerization I |
| | c | H45 Ferm | 8 | Fermentation without dilution |
| | d | H45 Ferm | 12 | Fermentation without dilution |
| | e | H45 Ferm | 8 | Sugar cane juice added |
| 33 | a | H46 | 12 | Hydrolyzed |
| | b | H46D | .33 | Depolymerization I |
| | c | E46 Ferm pH 3.5 | 4 | Fermentation without dilution |
| | d | E46 Ferm pH 3.5 | 8 | Fermentation without dilution |
| | e | E46 Diluted Ferm pH 3.5 | 8 | Fermentation with dilution 1:2, pH 3.5 |
| | f | Washing Water | | Pretreated bagasse |
| 34 | a | E48 | 12 | Hydrolyzed |
| | b | E48 DP | .33 | Depolymerization I |
| | c | E48 Ferm pH 3.5 | 4 | Fermentation without dilution |
| | d | E48 Ferm pH 3.5 | 8 | Fermentation without dilution |
| | e | E48 Diluted Ferm pH 3.5 | 8 | Fermentation with dilution 1:2, pH 3.5 |
| | f | E48 Diluted Ferm pH 4.5 | 8 | Fermentation with dilution 1:2, pH 4.5 |
| | g | E48 Diluted pH 4.5 | 8 | Fermentation with dilution 1:2, pH 4.5 |
| | h | Sugar cane juice Ferm | 4 | Fermentation without dilution |
| | i | Sugar cane juice Ferm | 8 | Fermentation without dilution |
| | j | Sugar cane juice Dil. | 8 | Fermentation with dilution 1:2, pH 4.5 |
| | | Ferm | | |
| 35 | a | E49 | 12 | Hydrolyzed |
| | b | E49 DP | .33 | Depolymerization I |
| | c | E49 Ferm pH 3.5 | 4 | Fermentation without dilution |
| | d | E49 Ferm pH 3.5 | 8 | Fermentation without dilution |
| | e | E49 Diluted Ferm pH 3.5 | 4 | Fermentation with dilution 1:2, pH 3.5 |
| | f | E49 Diluted Ferm pH 3.5 | 8 | Fermentation with dilution 1:2, pH 3.5 |
| | g | E49 Diluted Ferm pH 4.5 | 8 | Fermentation with dilution 1:2, pH 4.5 |
| | h | E49 Diluted Ferm pH 4.5 | 12 | Fermentation with dilution 1:2, pH 4.5 |
| | i | Media 48-53 | 12 | Fermentation of mixture of experiments |
| | | | | 48 a 53. |
| 36 | a | E50 | 12 | Hydrolyzed |
| | b | E50 DP | .33 | Depolymerization I |
| | c | E50 Ferm pH 3.5 | 4 | Fermentation without dilution pH 3.5 |
| | d | E50 Ferm pH 3.5 | 8 | Fermentation without dilution pH 3.5 |
| | e | E50 Diluted Ferm pH 3.5 | 4 | Fermentation with dilution 1:2, pH 3.5 |
| | f | E50 Diluted Ferm pH 3.5 | 8 | Fermentation with dilution 1:2, pH 3.5 |
| | g | E50 Diluted Ferm pH 4.5 | 8 | Fermentation with dilution 1:2, pH 4.5 |
| | h | E50 Diluted Ferm pH 4.5 | 12 | Fermentation with dilution 1:2, pH 4.5 |
| 37 | a | E51 | 12 | Hydrolyzed |
| | b | E51 DP | .33 | Depolymerization I |
| | c | E51 Ferm pH 3.5 | 4 | Fermentation without dilution pH 3.5 |
| | d | E51 Ferm pH 3.5 | 8 | Fermentation without dilution pH 3.5 |
| | e | E51 Diluted Ferm pH 3.5 | 4 | Fermentation with dilution 1:2, pH 3.5 |
| | f | E51 Diluted Ferm pH 3.5 | 8 | Fermentation with dilution 1:2, pH 3.5 |
| | g | E51 Diluted Ferm pH 4.5 | 8 | Fermentation with dilution 1:2, pH 4.5 |
| | h | E51 Diluted Ferm pH 4.5 | | Fermentation with dilution 1:2, pH 4.5 |
| 38 | a | E52 | 12 | Hydrolyzed |
| | b | E52 DP | .33 | Depolymerization I |
| | c | E52 Diluted Ferm pH 4.5 | 8 | Fermentation with dilution 1:2, pH 4.5 |
| 39 | a | E53 | | Hydrolyzed |
| | b | E53 DP | | Depolymerization I |
| | c | E52 Diluted Ferm pH 4.5 | 8 | Fermentation with dilution 1:2, pH 4.5 |

FIG. 40 shows a schematic representation of the methods of the present invention, which can be performed sequentially or separately, indicated respectively in 10, 20, 30 and 40: the reactor for the production of hydrolytic enzymes, the equipment for enzymatic hydrolysis, the equipment for chemical hydrolysis, and the reactor for the production of ethanol.

### DETAILED DESCRIPTION OF THE INVENTION

The present invention, among other developments, discloses innovative methods of fermenting the fungus Penicillium echinulatum strain 9A02S1 (DSM18942) in liquid state and/or in solid state for producing cellulases and/or hemicellulases. The method of the invention is innovational and is designed to fill in gaps found in the state of the art, presenting various alternatives for producing these enzymes, using substrates (inductive substances) and low-cost techniques, providing a highly efficient process of producing cellulases and hemicellulases, and distinguishing this technology from other existing forms.

In one embodiment of the present invention, fermentation processes in submerged culture and in solid state were used to produce xylanases and cellulases. In both processes, excellent enzymatic activity results were obtained.

The example below provides further details of the methodology used in this preferred embodiment of the invention:

### EXAMPLE 1

### Production of Cellulases and Hemicellulases by Submerged Fermentation

In the submerged culture for the production of cellulase and/or xylanase, sugar cane bagasse (in natura and pretreated) was used in order to lower the production cost of cellulases and xylanases and to add value to this residue which is so abundant in Brazil and in other countries. In the production of these enzymes, P. echinulatum mutants developed over many years were used. This fact constitutes novelty, because P. echinulatum mutants have not yet been used to produce cellulases and xylanases in sugar cane bagasse as an inductive source and source of carbon.

To produce cellulases and xylanases in submerged culture, initially the sugar cane bagasse is ground and chemically pretreated with pretreatment solutions formulated with sodium hydroxide, hydrogen peroxide and anthraquinone (Tables 3 and 4).

TABLE 3
Formulation of pretreatment solutions.
Pretreatment solutions

| | |
|---|---|
| S1 | 16% NaOH |
| S2 | 16% NaOH + 0.3% H₂O₂ |
| S3 | 16% NaOH + 0.3% H₂O₂ + 0.02% AQ |
| S4 | 16% NaOH + 0.3% H₂O₂ + 0.02% AQ + 0.3% EDTA |
| S5 | 16% NaOH + 0.6% H₂O₂ |
| S6 | 16% NaOH + 0.6% H₂O₂ + 0.02% AQ |
| S7 | 16% NaOH + 0.6% H₂O₂ + 0.02% AQ + 0.3% EDTA |

NaOH: sodium hydroxide;
AQ: anthraquinone
H₂O₂: hydrogen peroxide

TABLE 4
Pretreatments of sugar cane bagasse.

| | Solution employed |
|---|---|
| | (proportion of solution:sugar cane |
| Pretreatment | bagasse) |
| T1 | S1 - 6:1 |
| T2 | S1 - 3:1 |
| T3 | S2 - 6:1 |
| T4 | S2 - 3:1 |
| T5 | S3 - 6:1 |
| T6 | S3 - 3:1 |
| T7 | S4 - 6:1 |
| T8 | S4 - 3:1 |
| T9 | S5 - 6:1 |
| T10 | S5 - 3:1 |
| T11 | S6 - 6:1 |
| T12 | S6 - 3:1 |
| T13 | S7 - 6:1 |
| T14 | S7 - 3:1 |
| T15 | Untreated bagasse (bnt) |
| T16 | Cellulose (Control) |

The mixture obtained in the prior stage (sugar cane bagasse and pretreatment solution) is submitted to temperatures in the range of 100-120° C., under pressure of 1 atmosphere of pressure for 15-20 minutes or this mixture remains from 1 to 3 days at ambient temperature. Thereafter, the lignin is removed and this pretreated bagasse is used in the production of the enzymes—cellulases and xylanases. Accordingly, mineral salts, soy bran and distilled water are added. This mixture is autoclaved at 121° for 20 minutes, then inoculated with 10⁵ spores of P. echinulatum per milliliter of culture medium or with 10% of final volume with this fungus culture previously grown for 48 hours. The submerged cultures are kept at a temperature of 28° C., for up to four days. The duration time of the culture depends on the enzyme of the enzymatic complex for which a greater proportion is desired. For example, if the intention is to produce a juice with higher xylanase activity, the method is interrupted on the 2^{nd} day of culture. However, if a higher endoglycanase activity is required, the method is interrupted on the 3^{rd} day, and Filter Paper Activity or β-glycosidases enzymes are desired, the collection should be performed on the 4^{th} or 5^{th} day.

To collect the enzymes, the culture is filtered to remove the mycelium and the enzymatic juice is harvested. Enzymatic doses of Filter Paper Activity, endoglycanases, β-glycosidades and xylanases are performed whereby to determine the precise quantity of enzymatic juice that is to be used for each application of these enzymes.

Another embodiment of the present invention employs the common inventive concept in the development of new industrial methods involving the hydrolysis of sugar cane bagasse. Next is a description of the industrial methods for producing hydrolytic enzymes, for the hydrolysis of sugar cane bagasse, for the production of additives comprising fermentable sugars and the respective additives, and methods of producing ethanol. The invention methods comprised the submerged culture of Penicillium echinulatum strain 9A02S1 (DSM18942), in substrates containing sugar cane bagasse and/or the use of enzymes of said microorganism in hydrolytic processes. Said fungus is a filamentous fungus that has been developed over the last 10 years at the University of Caxias do Sul by successive mutageneses and selections, having been identified as a good producer of cellulases and xylanases and deposited at the German resource center for biomaterial Deutsche Sammlung von Mikroorganismen und Zellkulturen (DSM) under number DSM18942, strain 9A02S1.

The methods of this invention are innovative and are designed to fill in various gaps found in the state of the art. Various alternatives are presented for the production of enzymes, the hydrolysis of sugar cane bagasse, the production of fermentable sugars and ethanol using substrates and low-cost techniques which make for highly efficient production methods, setting this technology apart from other current technologies.

For illustration purposes, but not to limit the scope of the invention, the examples below disclose methods in which the substrate—sugar cane bagasse—is used in natura and/or pretreated.

### EXAMPLE 2

### Method of Obtaining Hydrolytic Enzymes

The method of obtaining of the present invention comprises the submerged culture of Penicillium echinulatum strain 9A02S1 (DSM18942), in substrates containing sugar cane bagasse. Said method is carried out in industrial fermenters.

Pre-inoculum: Using the solid means stock in a test tube, the spores of the test tube are suspended in the culture medium. Table 5 shows the composition of a is preferred culture medium in the present invention. The volume of pre-inoculum used in the production fermenter may vary according to the characteristics of the equipment available and the desired process time. This present preferred embodiment uses pre-inocula corresponding to a 20% volume of the subsequent fermentation volume.

TABLE 5
composition of the preferred culture medium of pre-inoculum

| Raw Material | Concentration g/L |
|---|---|
| Bagasse pretreated by steam explosion | 20.00 |
| Saccharose | 1.00-2.50 |
| Soy protein | 1.00-2.50 |
| Ammonium sulfate | 1.00-2.80 |
| Urea | 0.30 |
| KH₂PO₄ | 1.50-3.00 |
| MgSO₄ | 0.30 |
| CaCl₂ | 0.30 |
| Gentamicin* | 0.010 |
| Twen 80 | 1.00-0.05 |
| Antifoaming | 1.00-0.05 |

### Growth Conditions and Fermentation

The suspension of spores from the test tube in a culture medium referred to above is aseptically introduced into culture flasks. The culture is taken to an incubation station with a shaker. The preferred culture conditions in this stage are: rotation at 120 rpm and temperature between 28-31° C. In reactors, preferred shaking is at 120 rpm for the first three inocula and at 70 rpm for the others, keeping the temperature between 28-30° C. and aeration from 1 to 0.7 vvm or 35% saturation of O₂.

### Cultivation Time and Collection of Samples:

In all the pre-inoculum conditions and the fermentation itself, the preferred culture time is 48 hours. For the inocula, the samples should be collected initially after 12 hours for observation of the presence or not of contaminants. In the event that contaminants are present, the control can be by choosing the pH, and it is advisable to reduce the pH level to 3 with phosphoric acid. If there is contamination in the pre-inoculum, it is advisable to reduce the pH level to 3 in the next medium that receives the inoculum. Samples of fermentation should be taken every 12 hours for analysis of microbial activity. Tests carried out under these conditions indicate that contamination control is effective, in various scales, thus constituting a major advantage in terms of method control on an industrial scale.

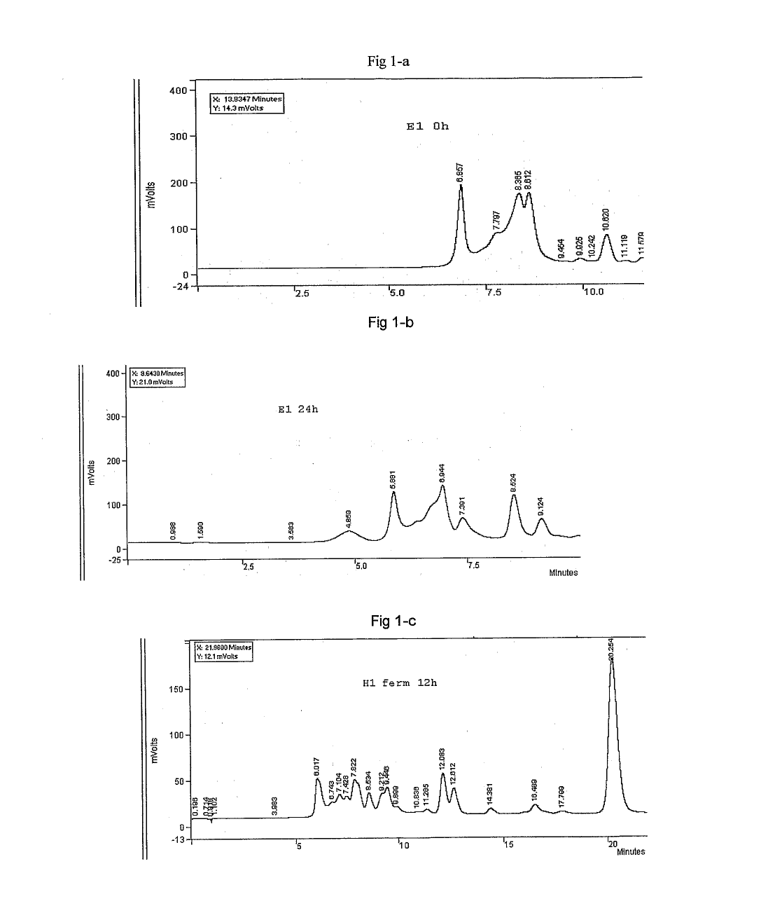

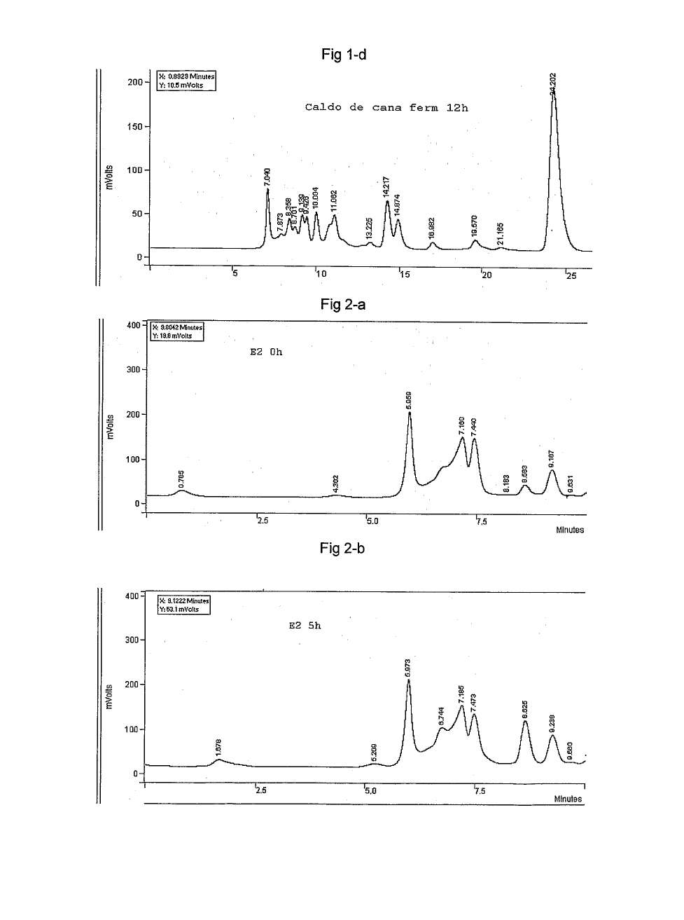

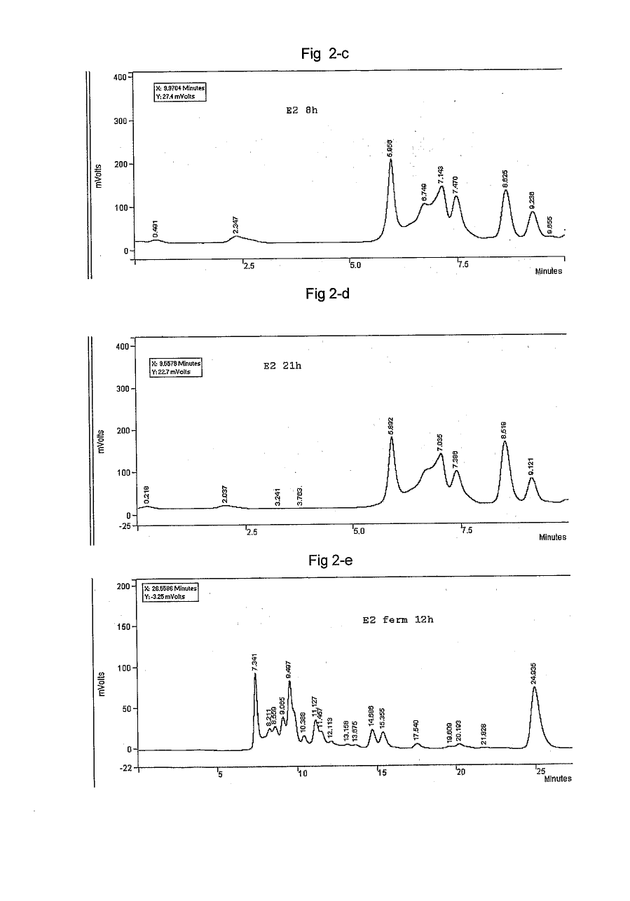

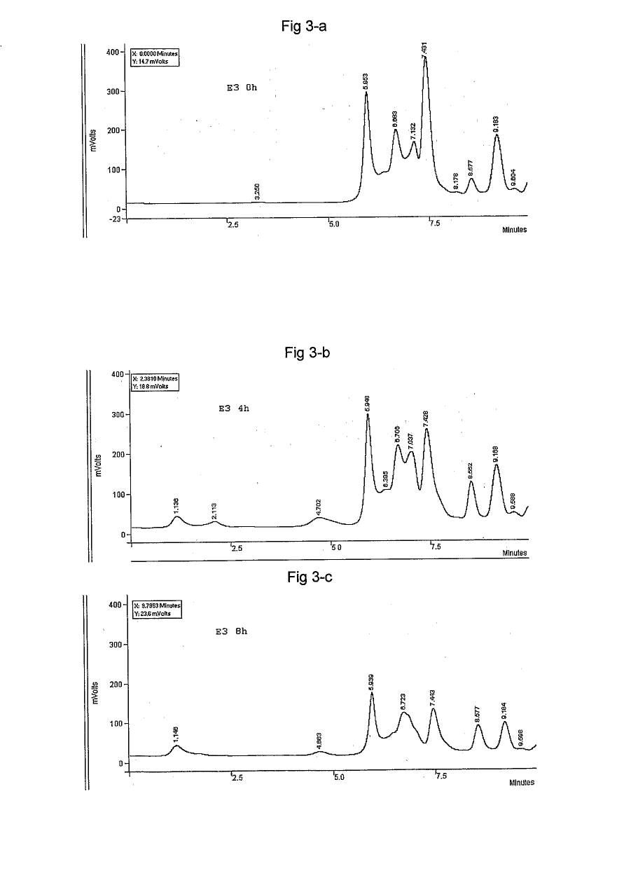

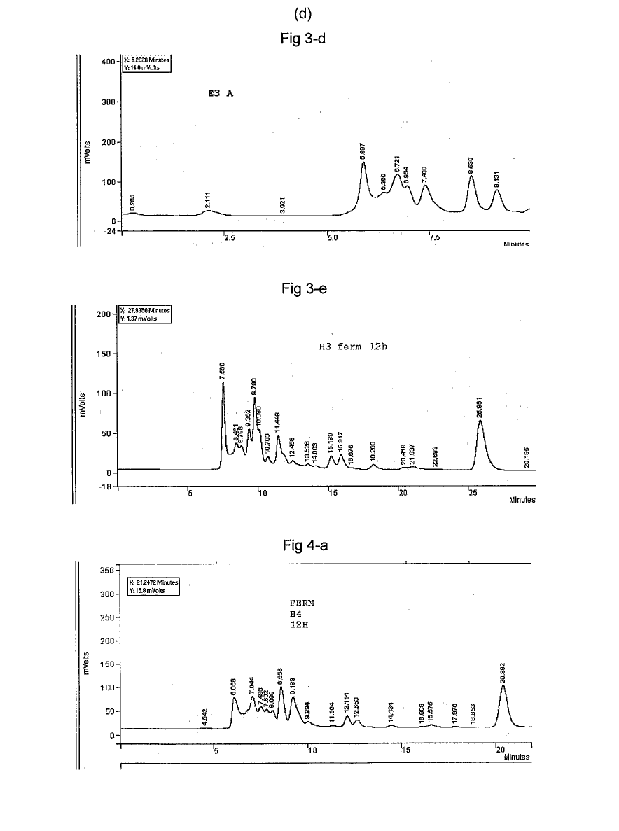

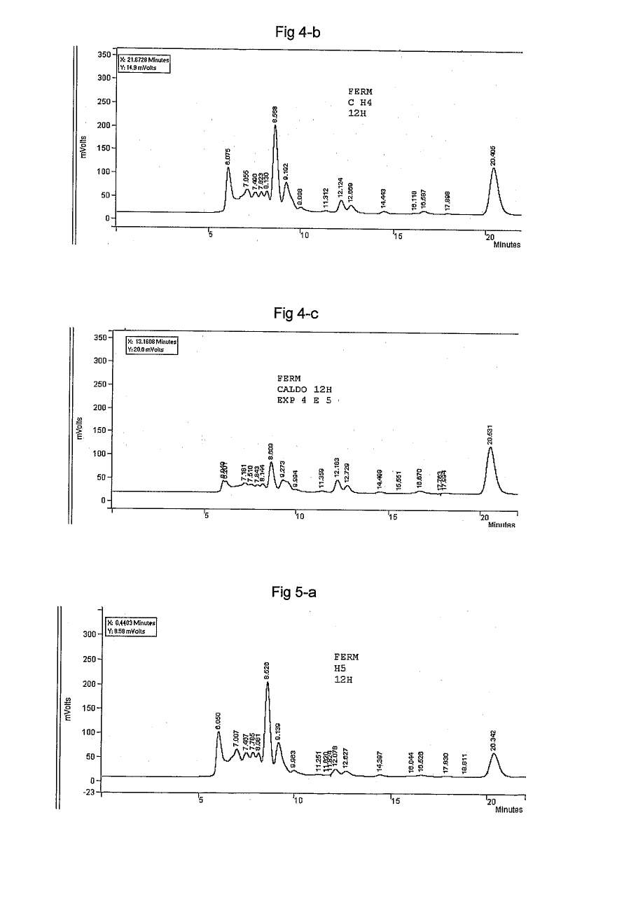

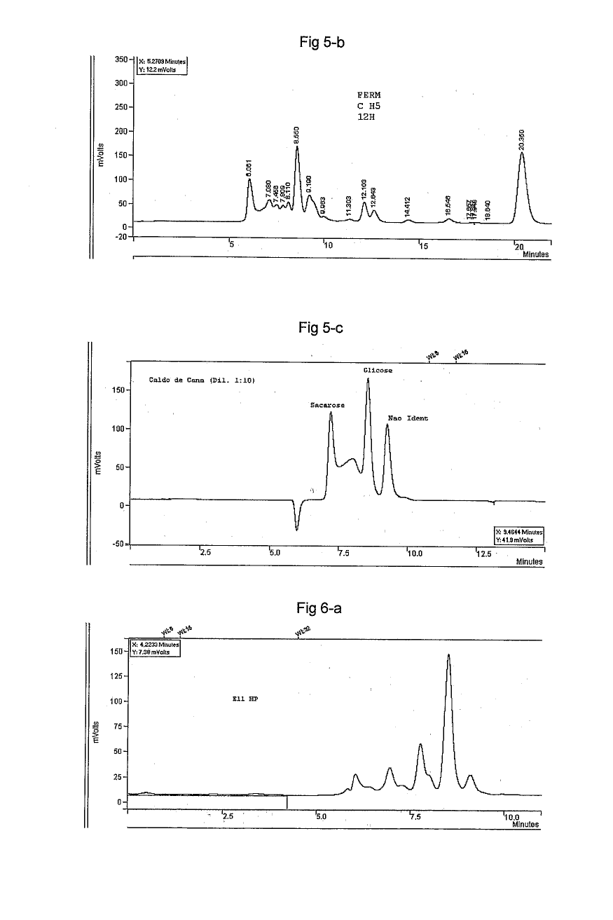

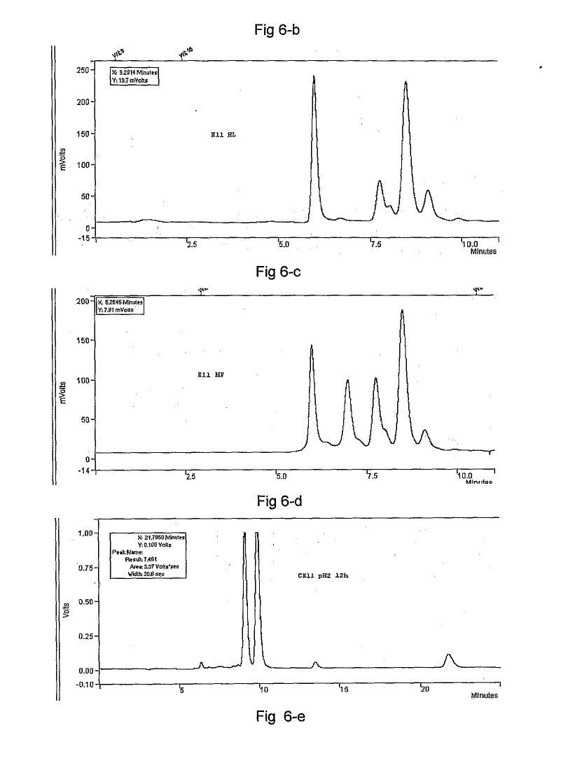

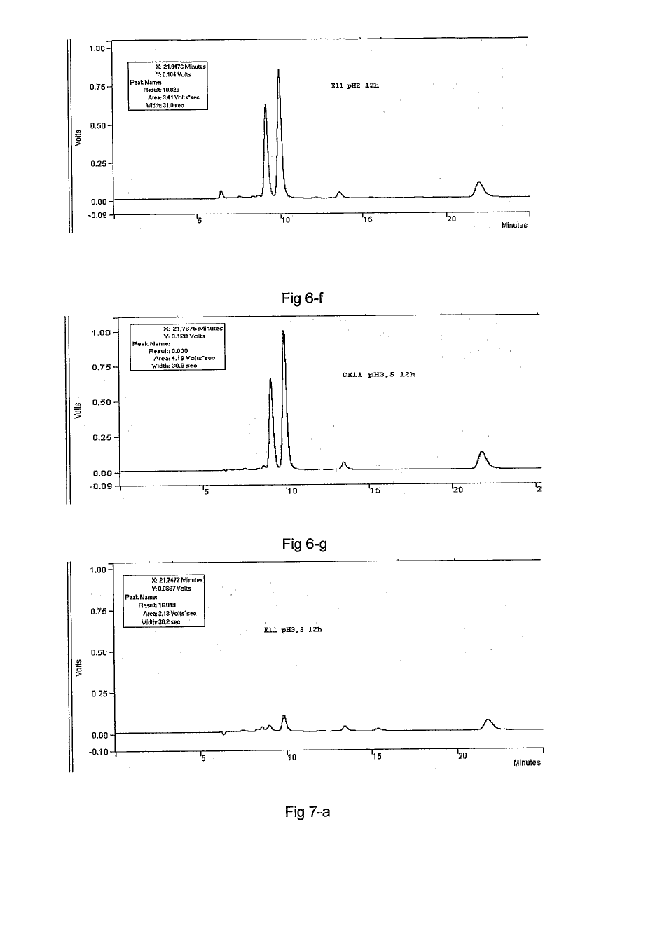

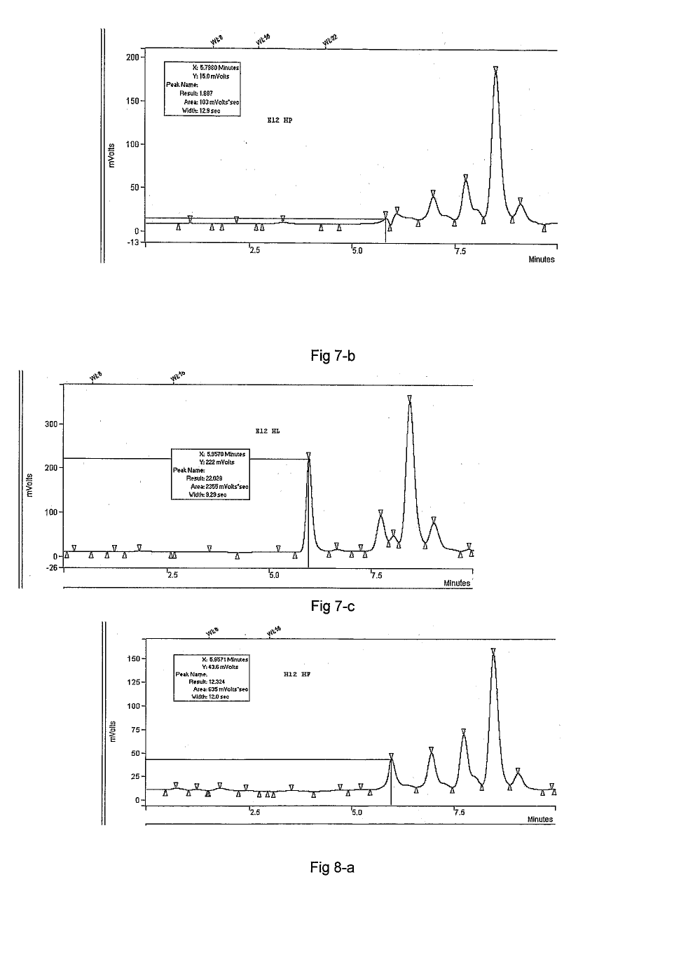

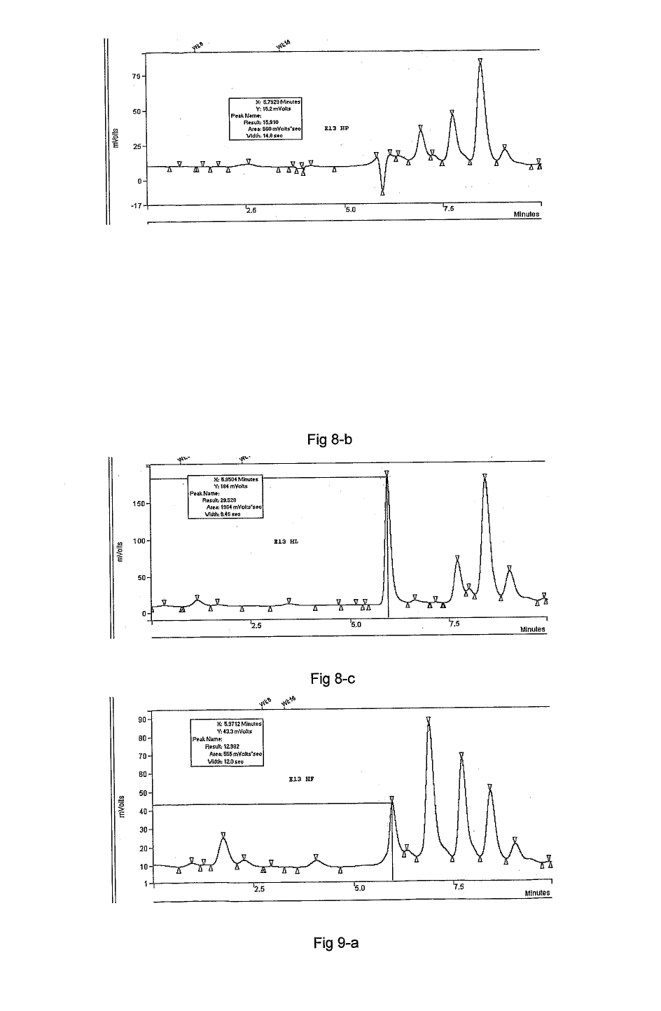

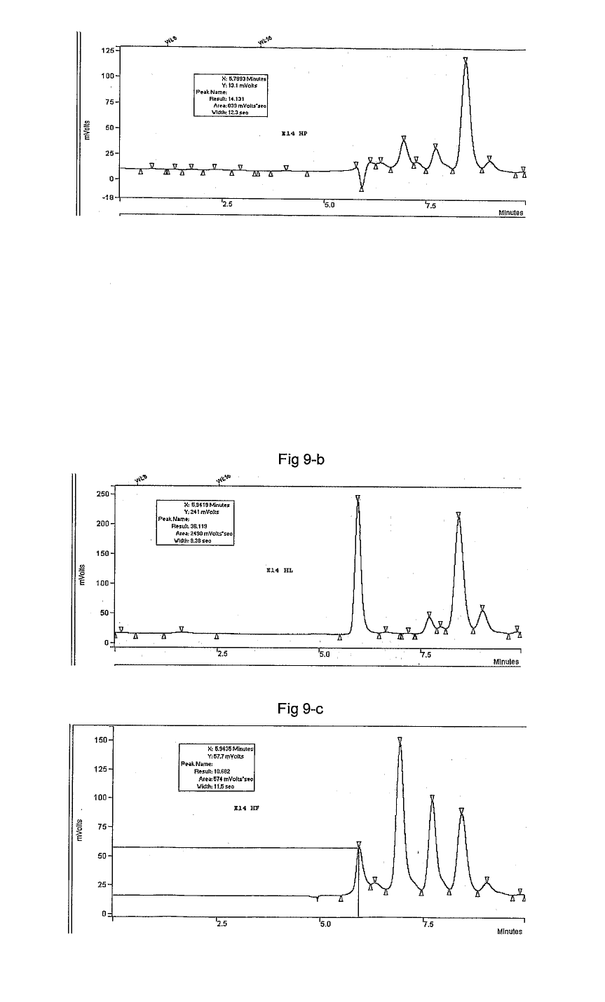

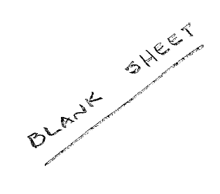

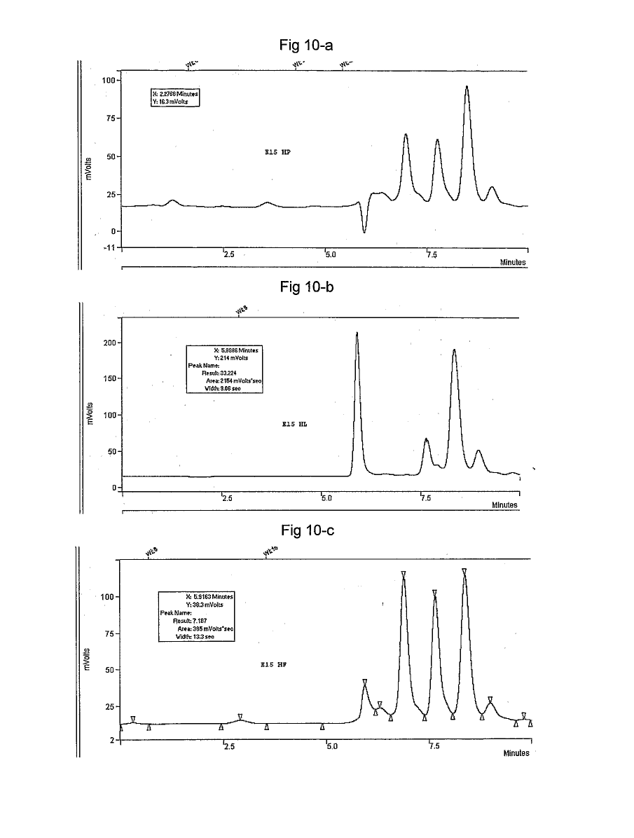

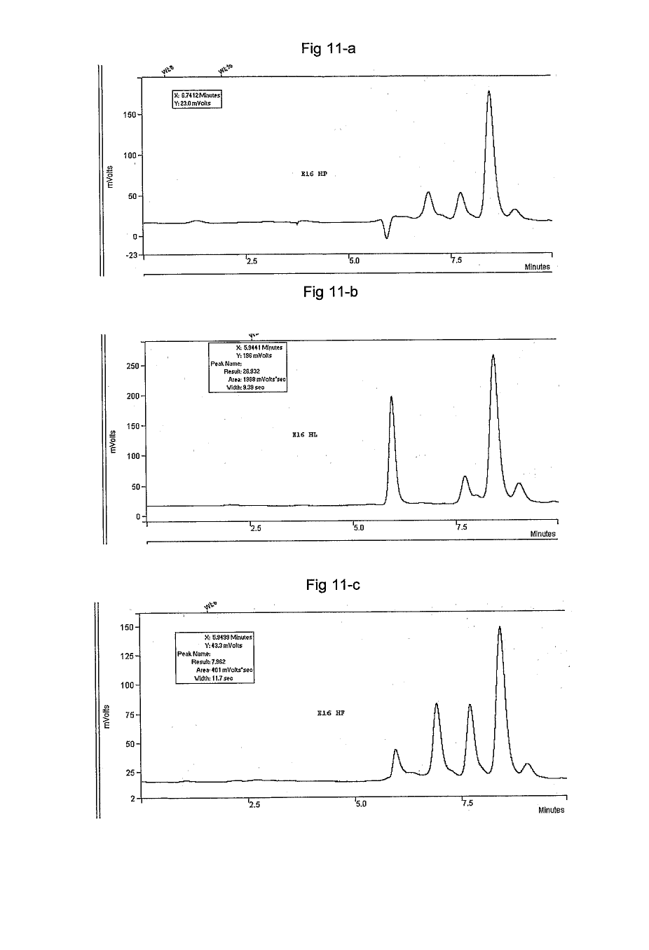

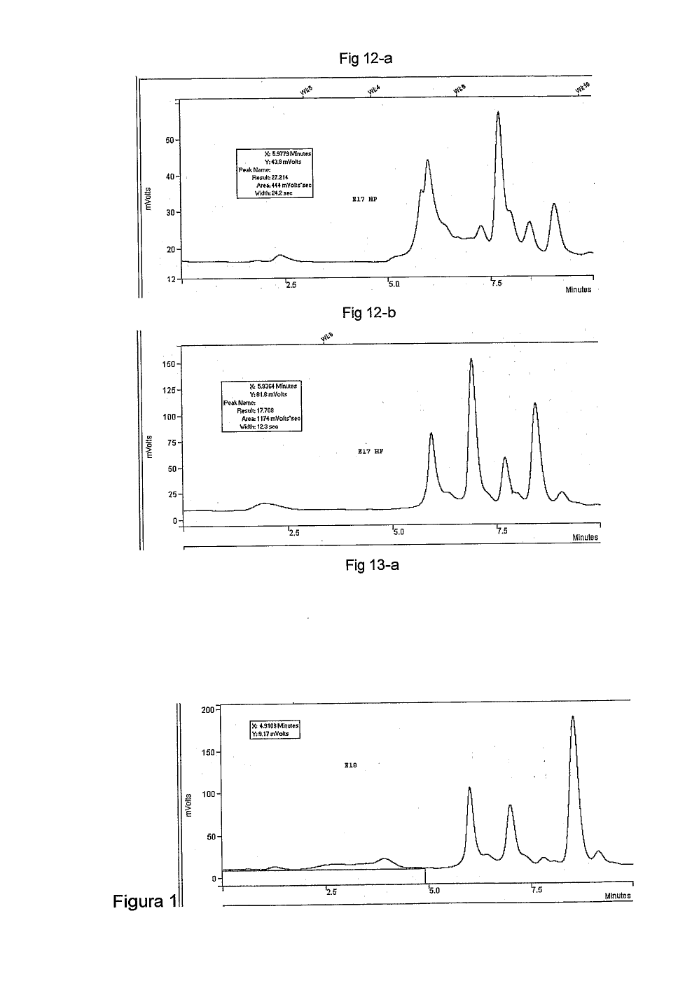

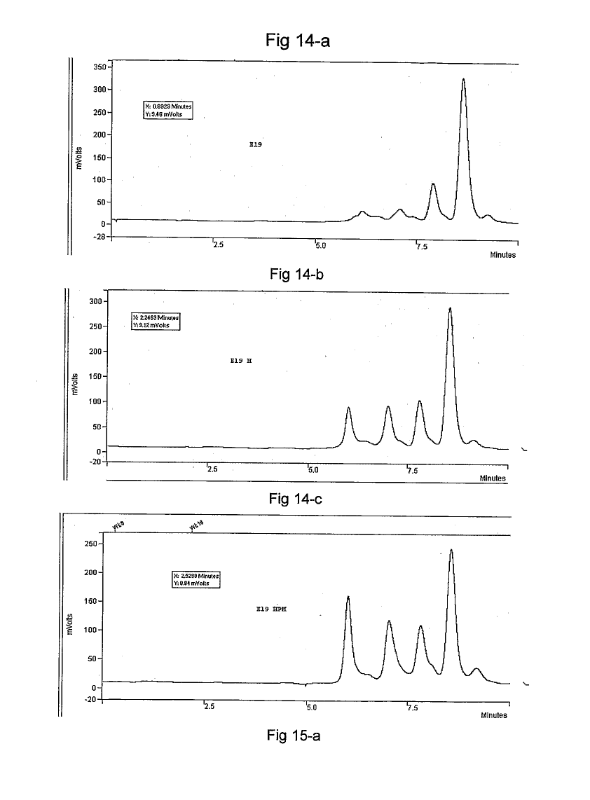

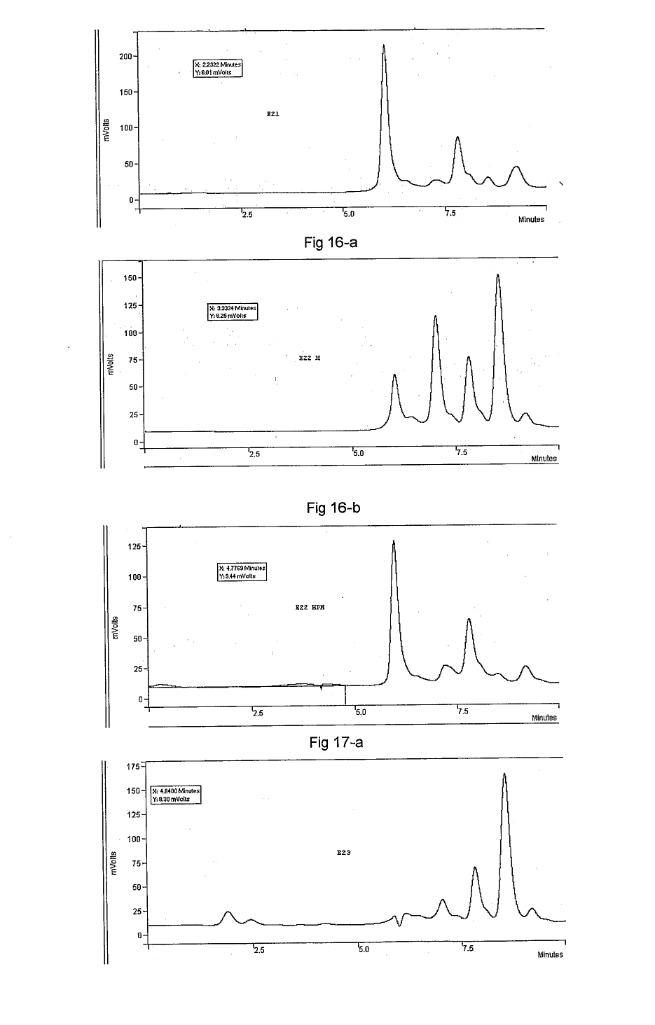

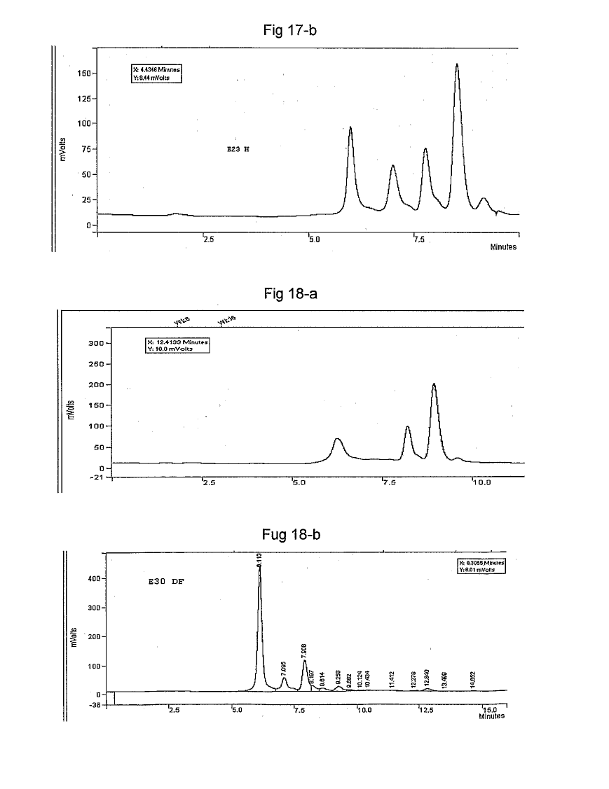

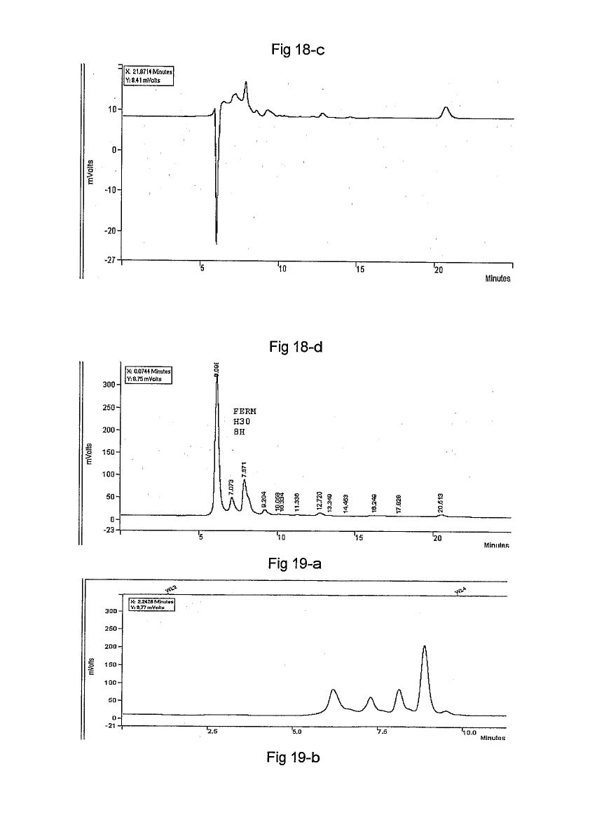

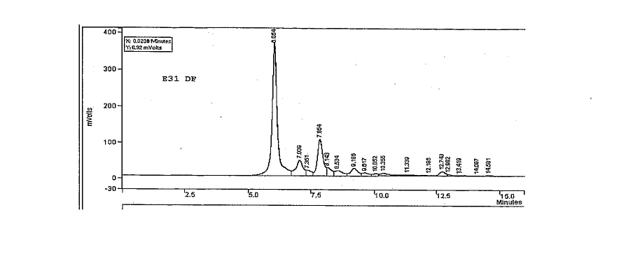

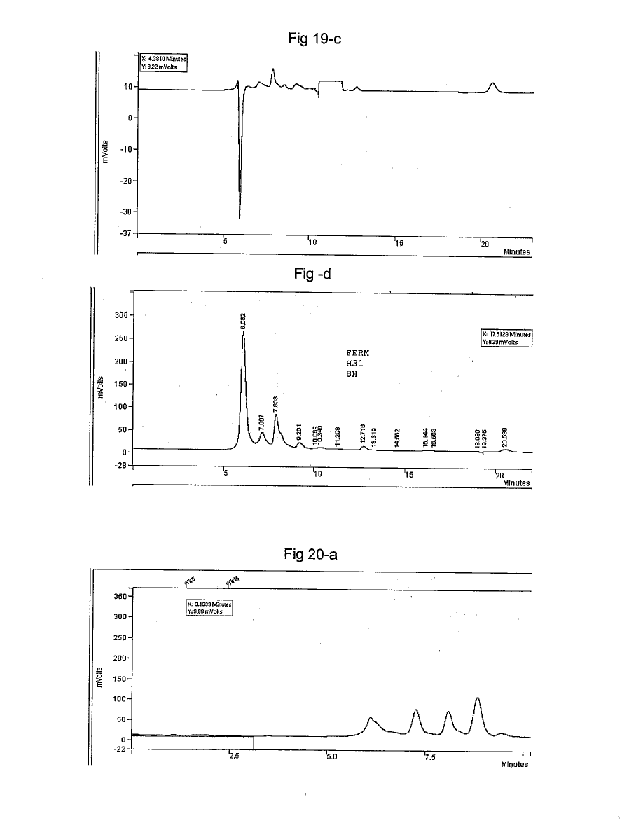

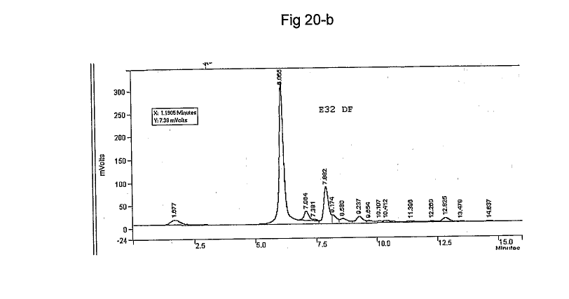

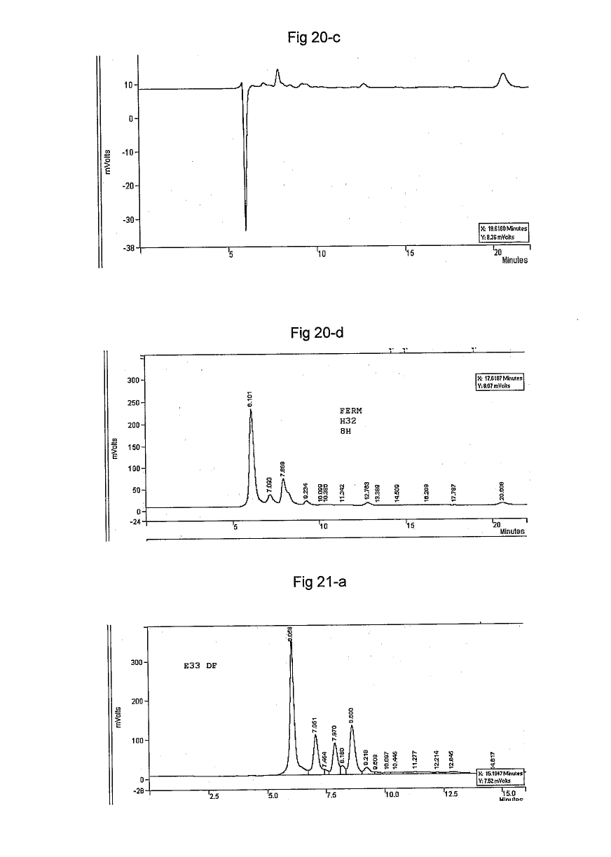

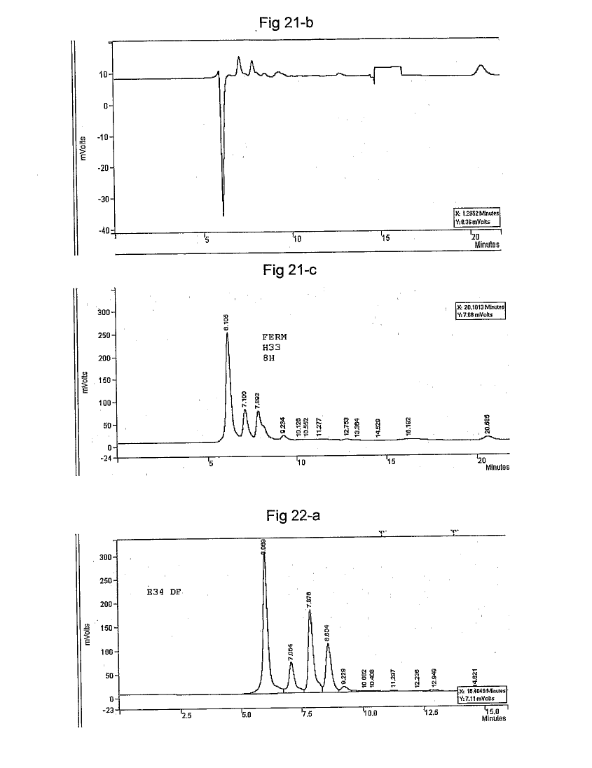

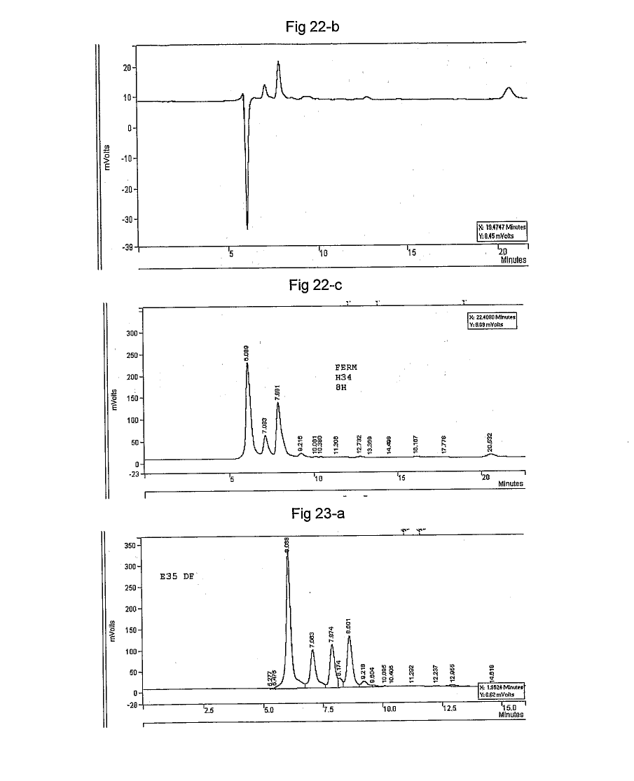

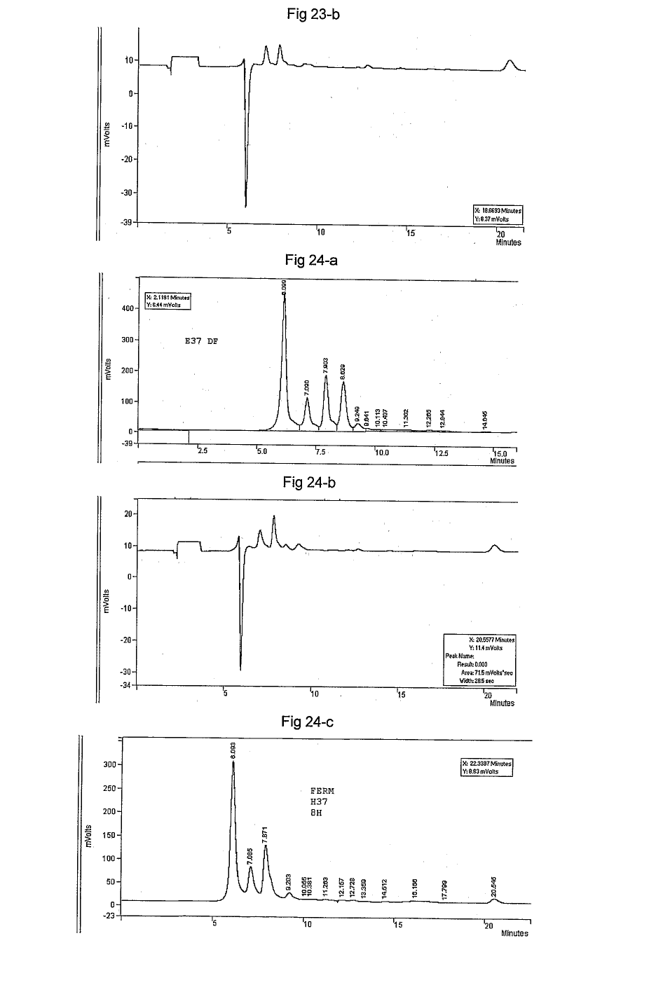

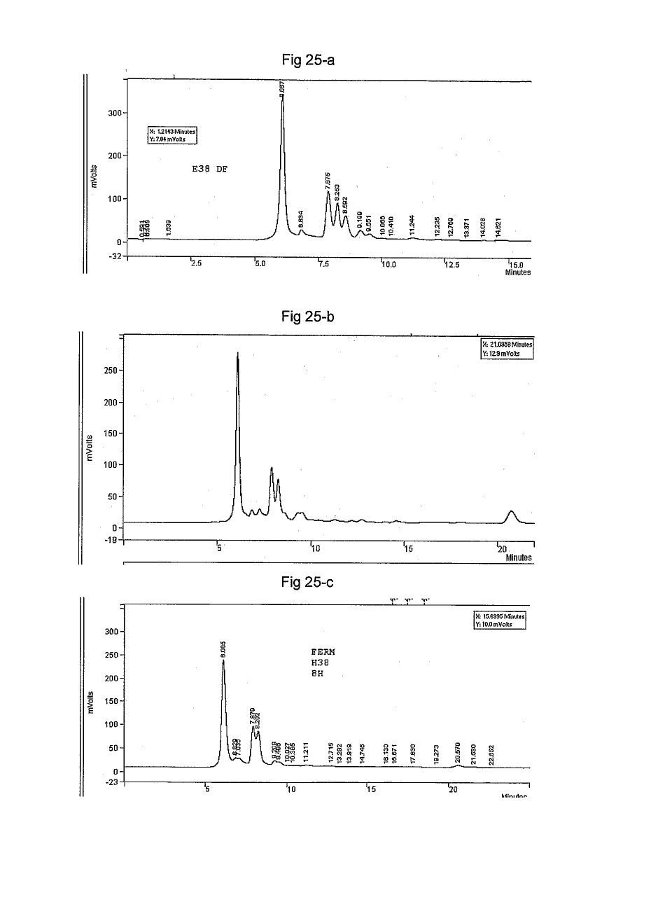

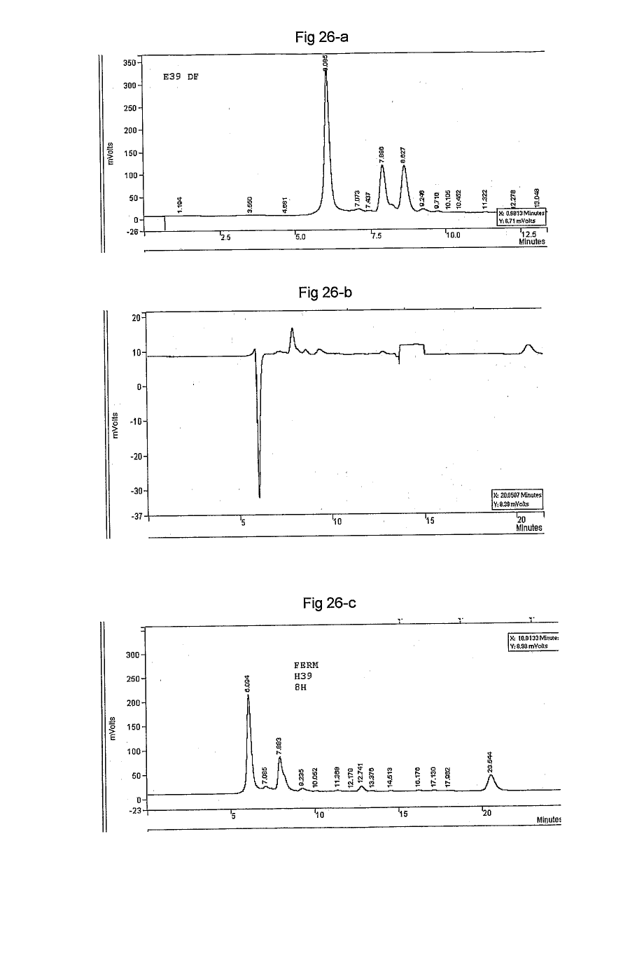

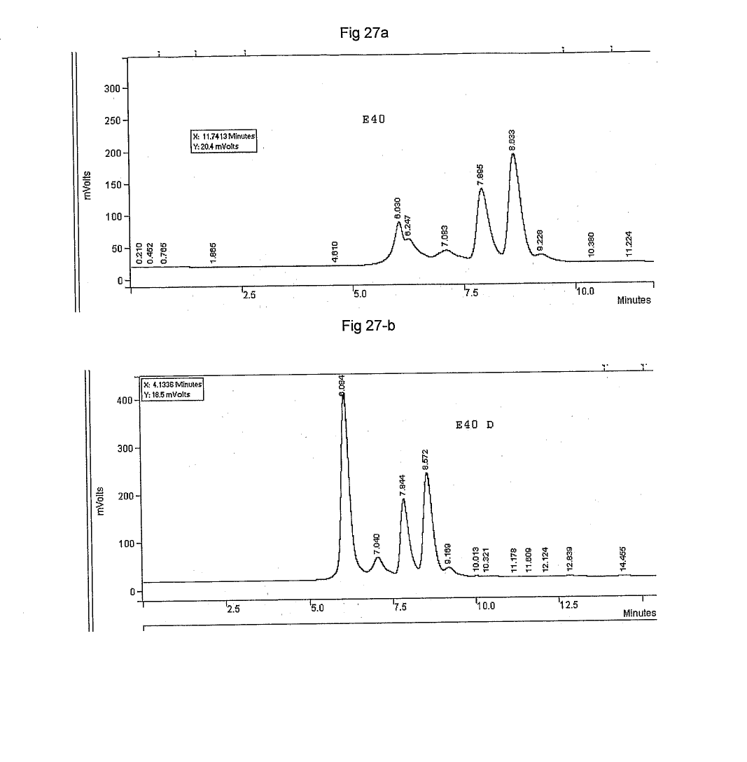

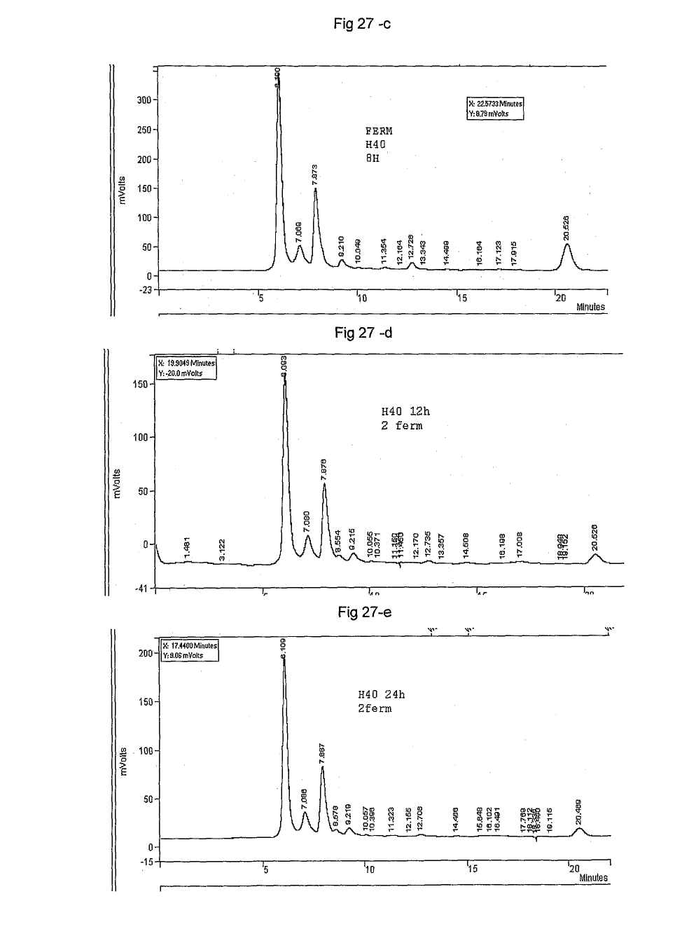

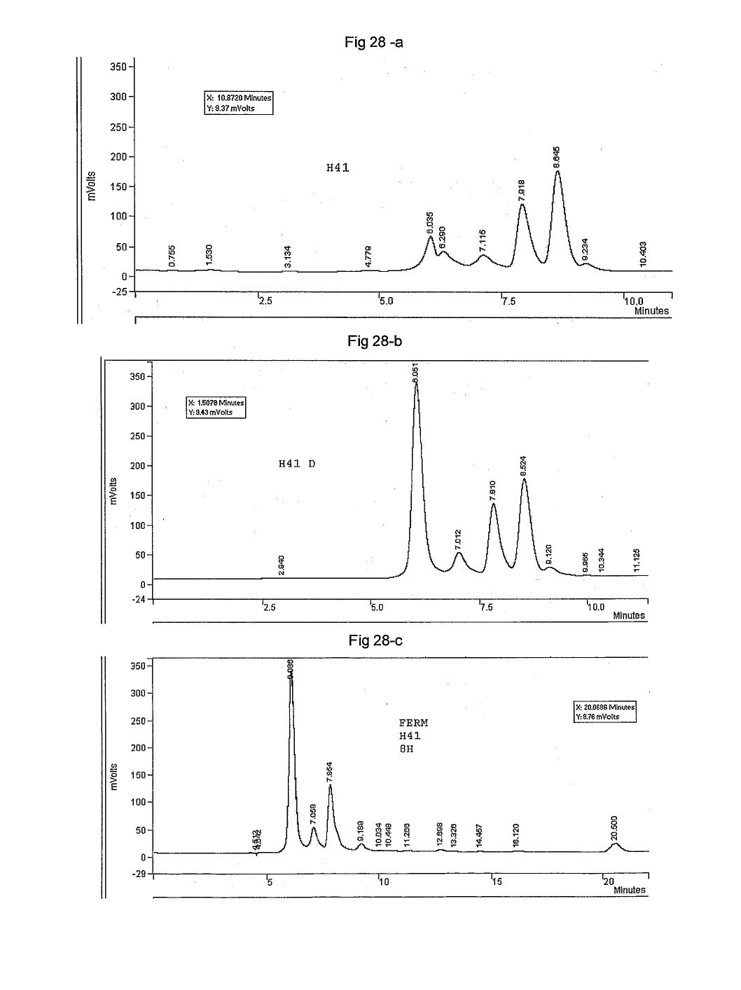

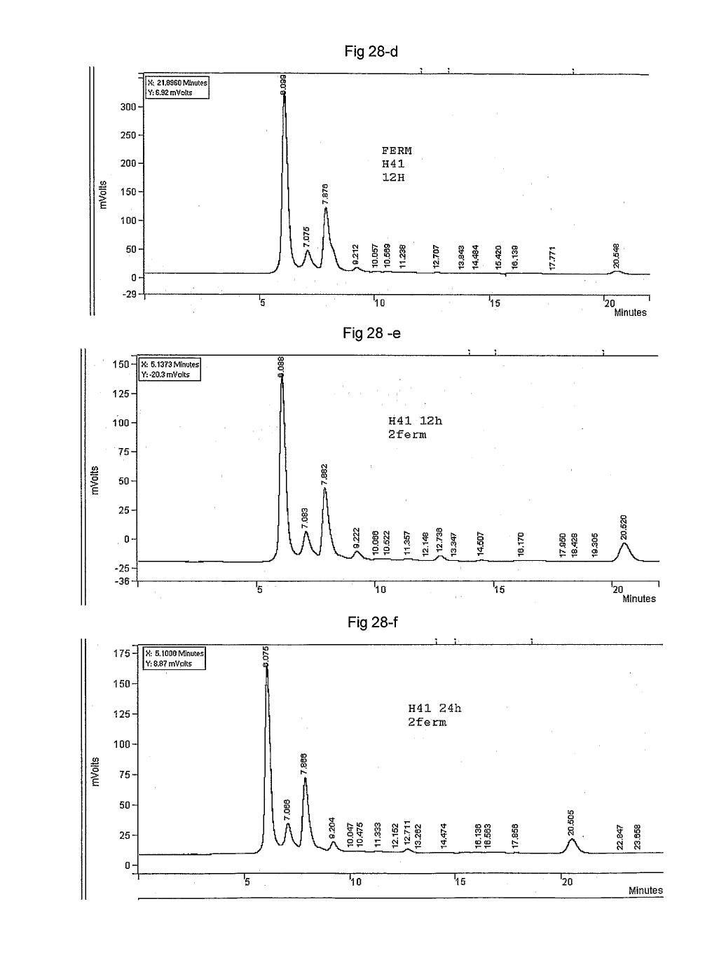

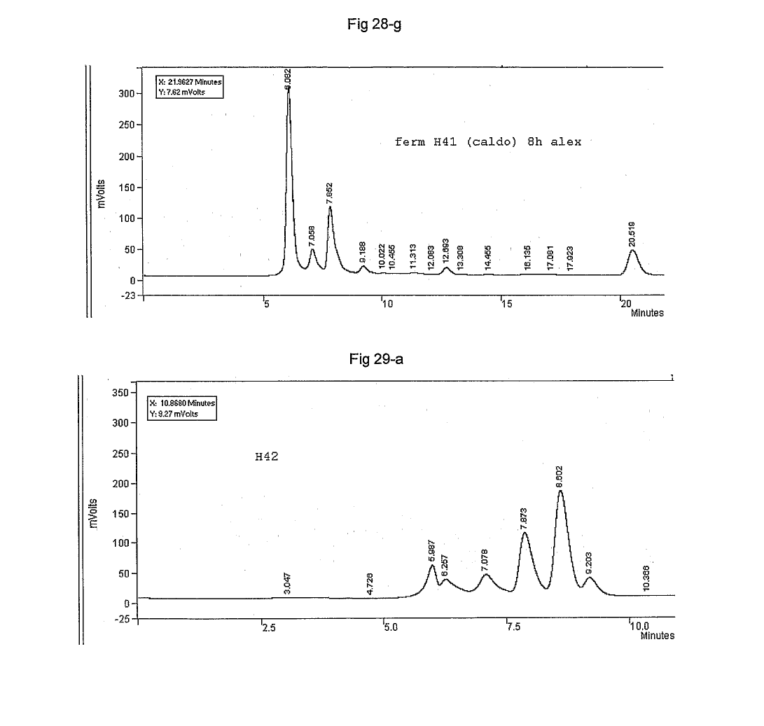

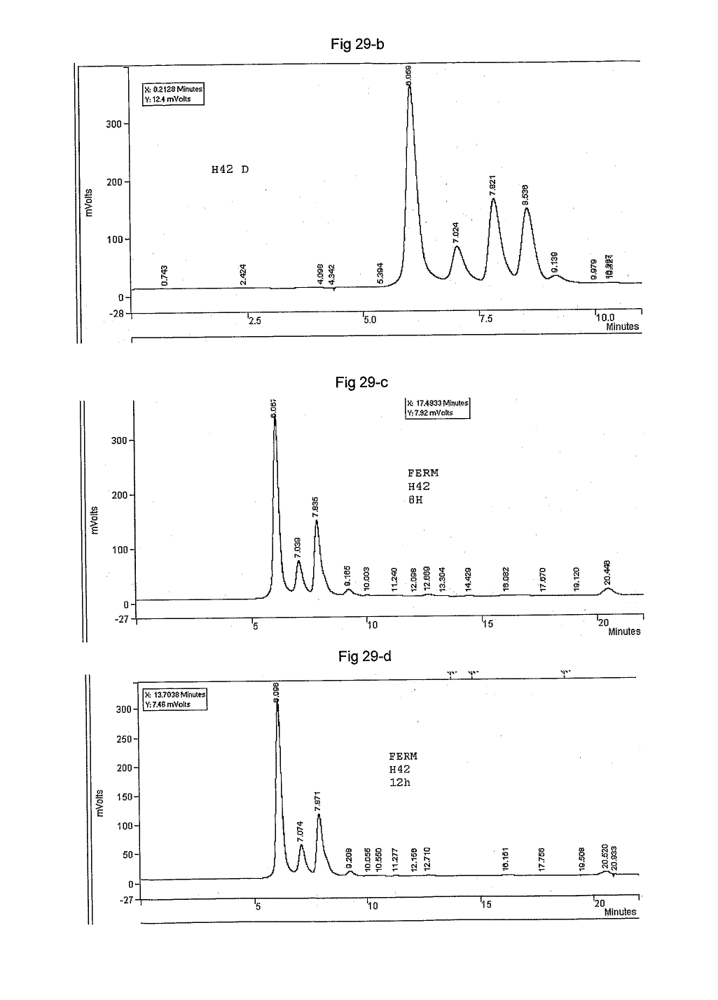

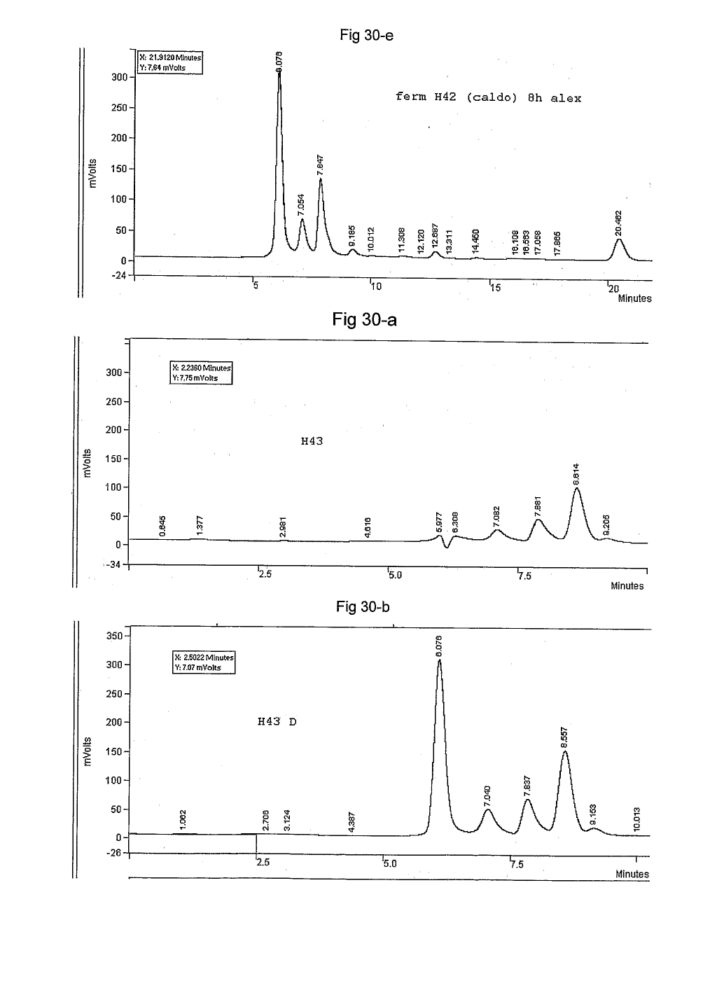

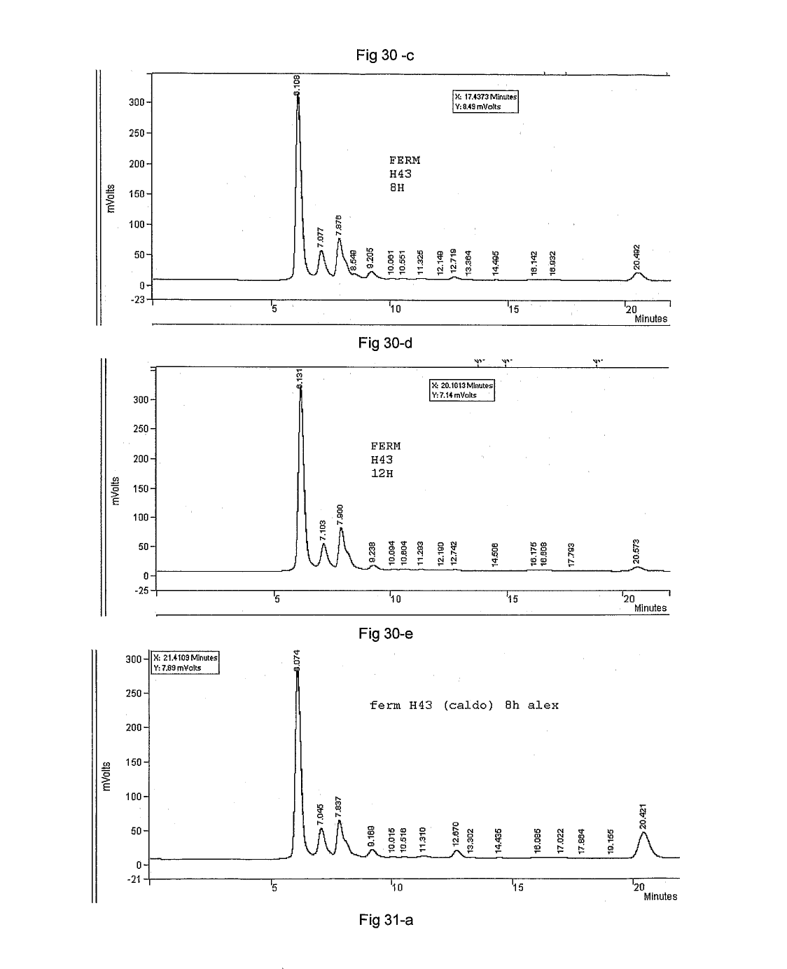

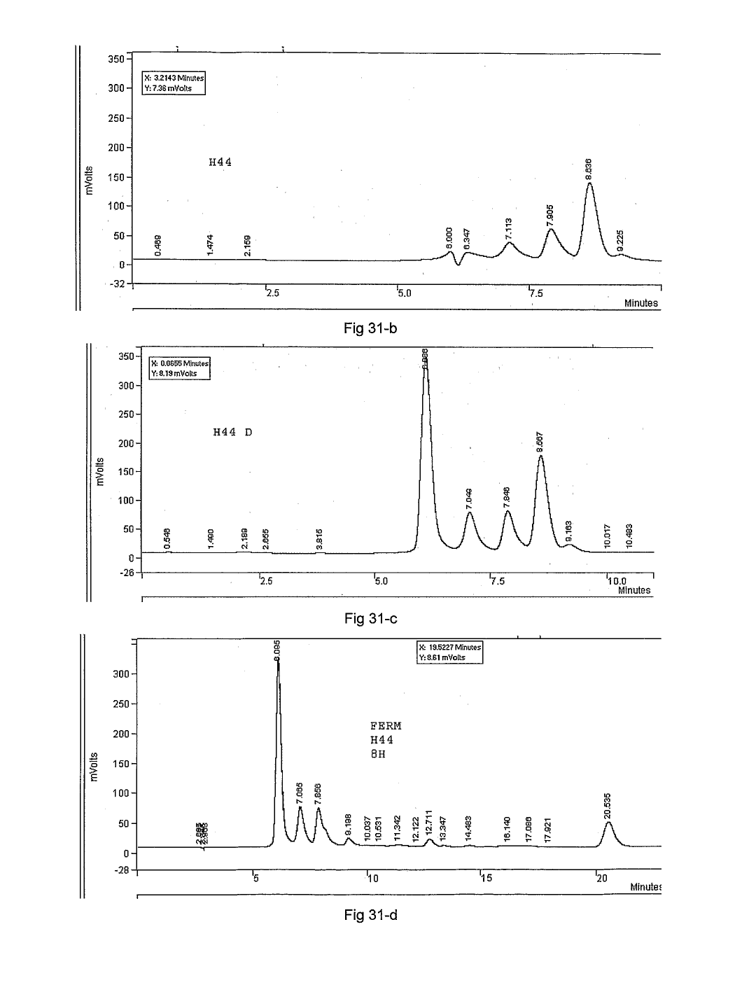

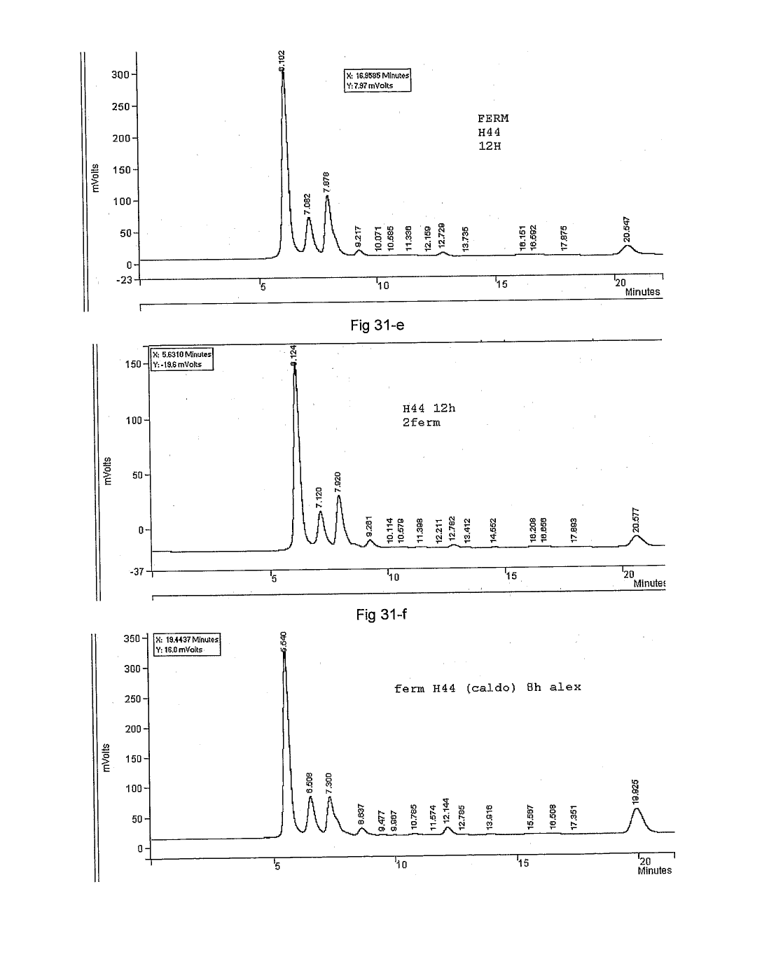

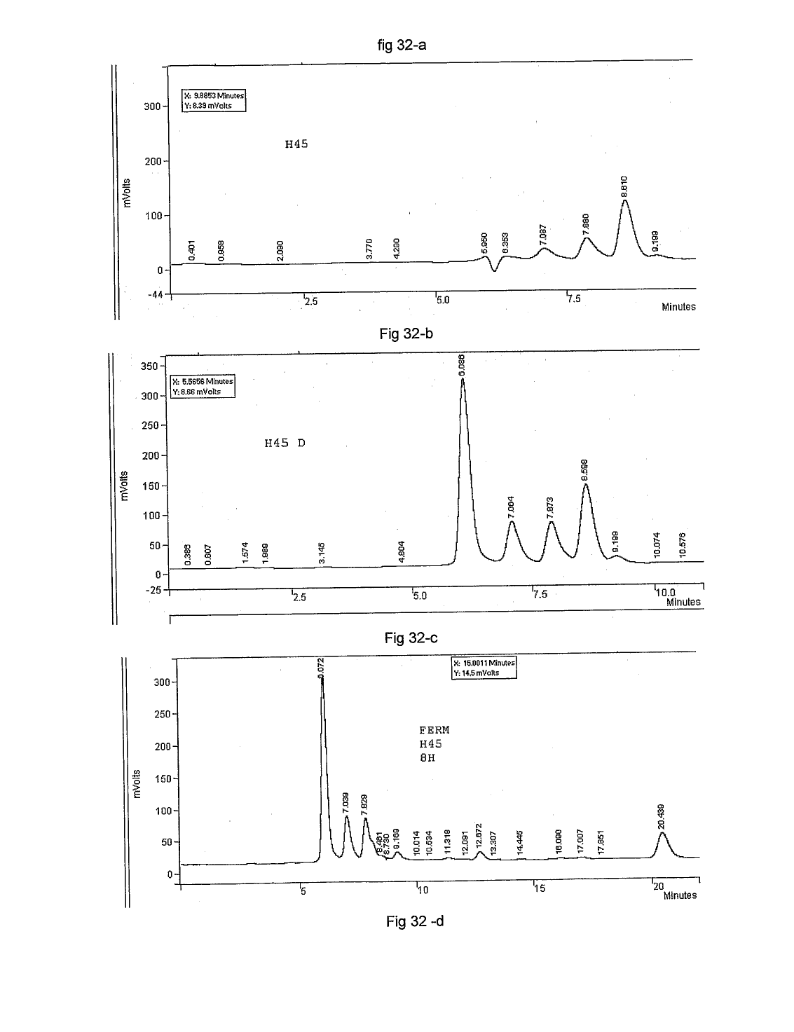

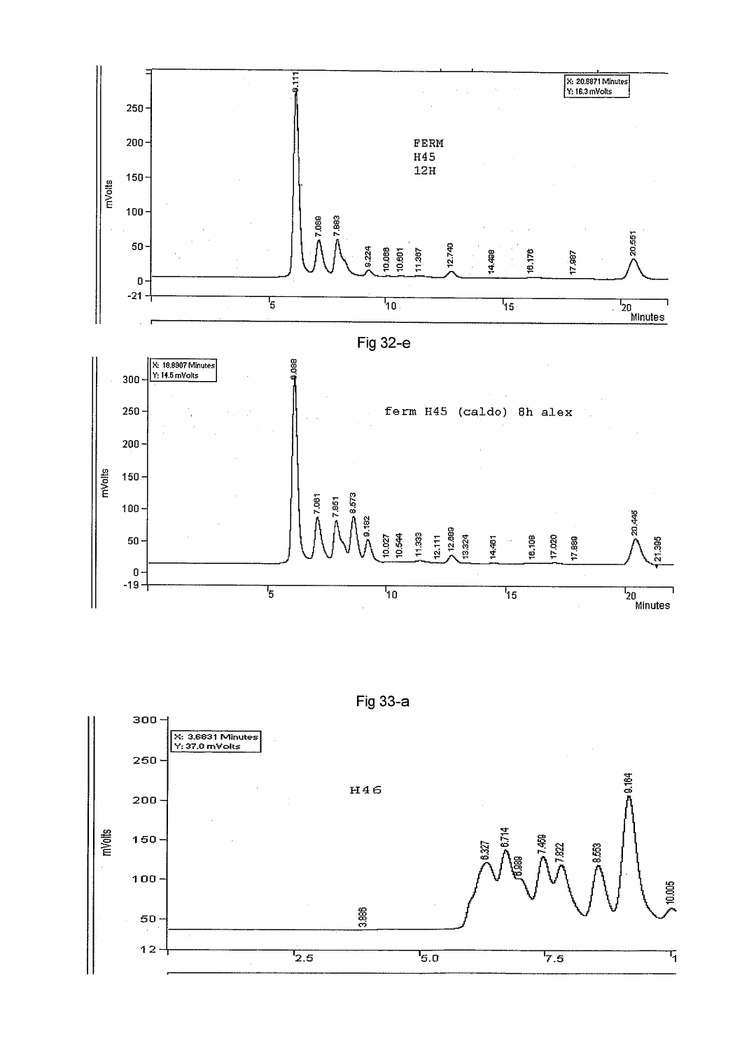

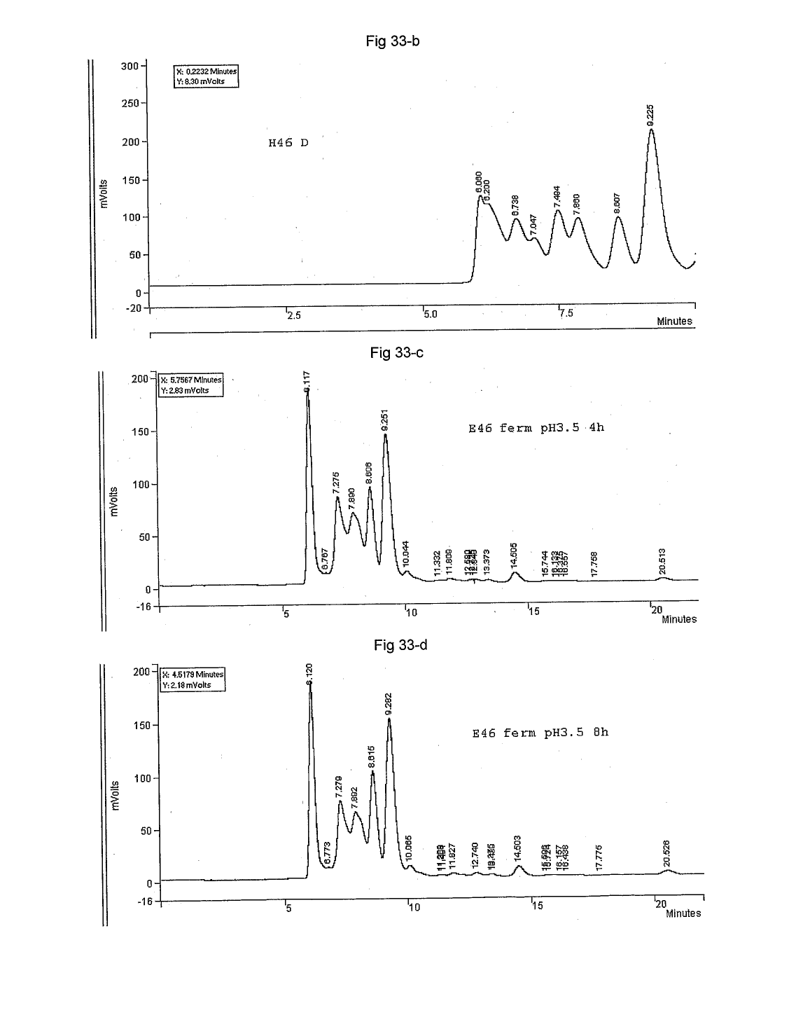

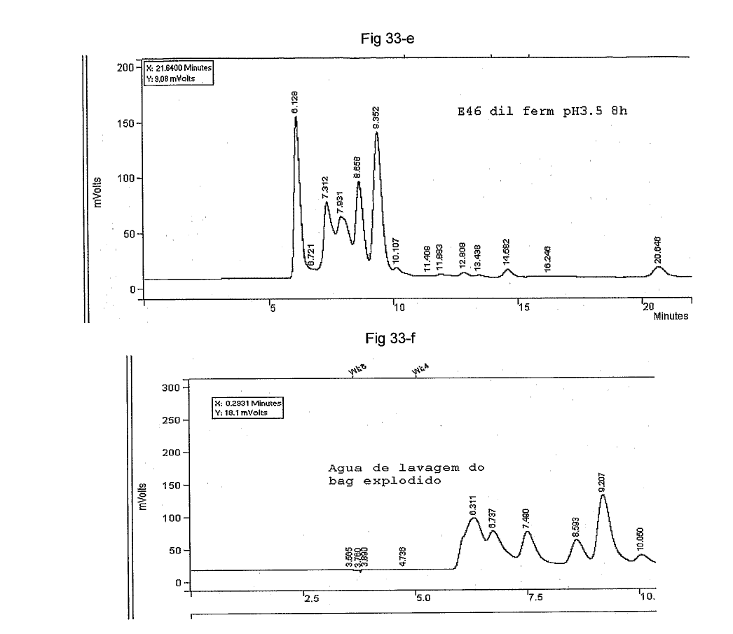

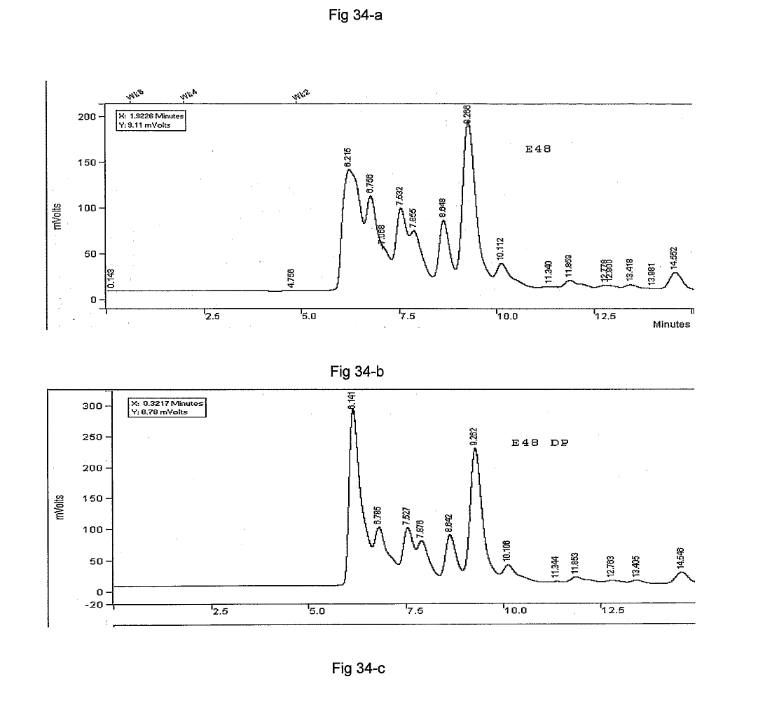

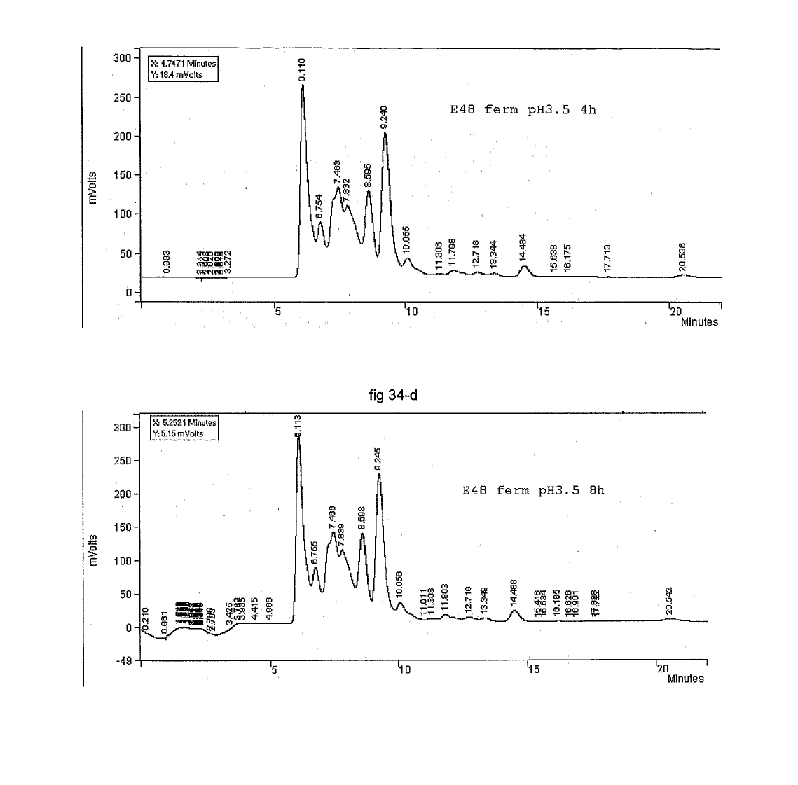

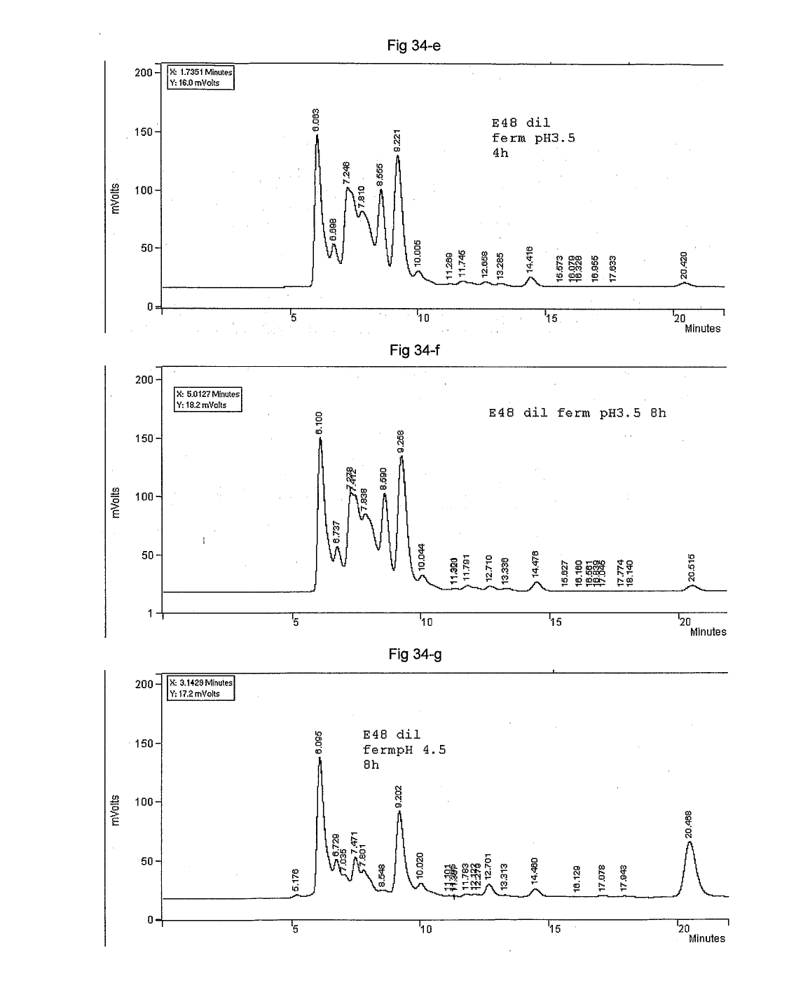

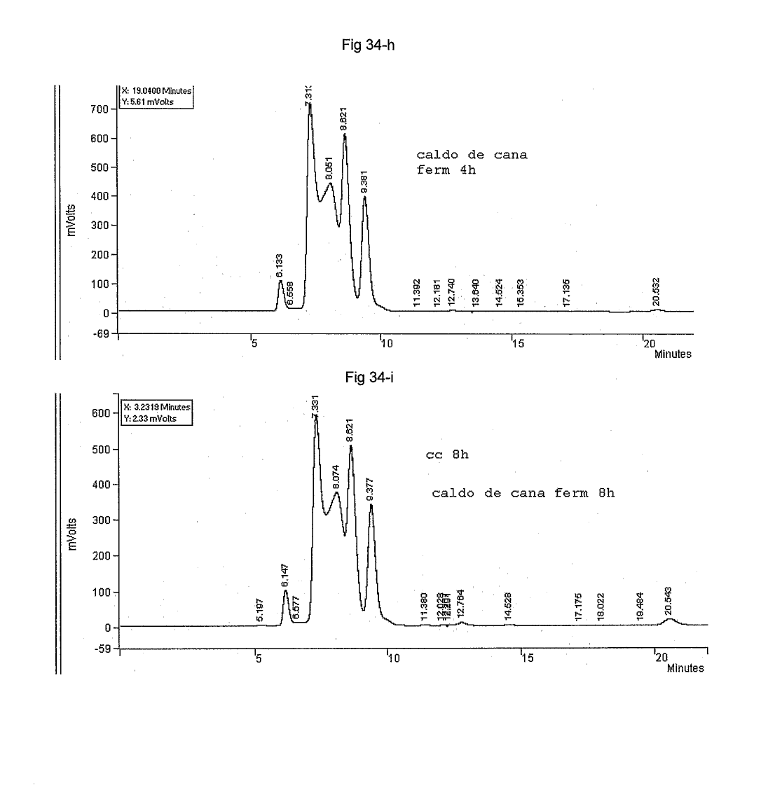

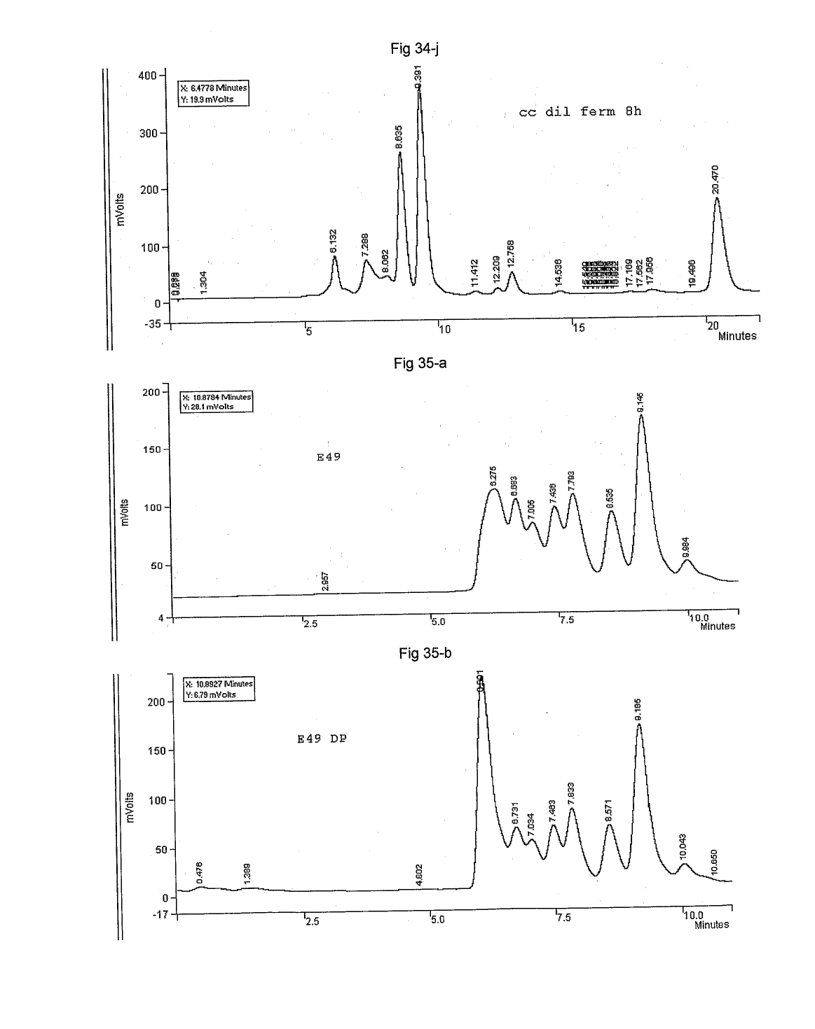

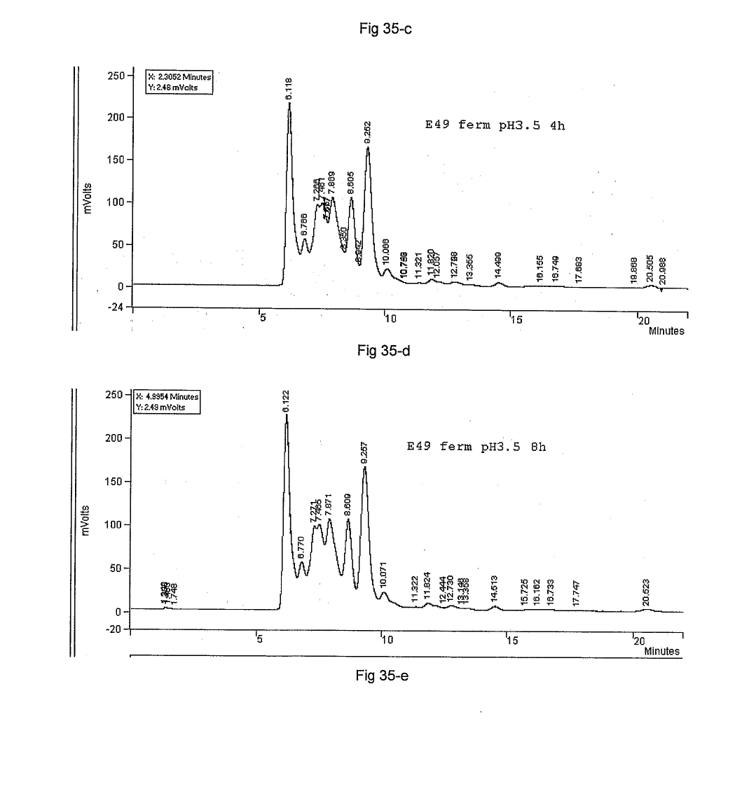

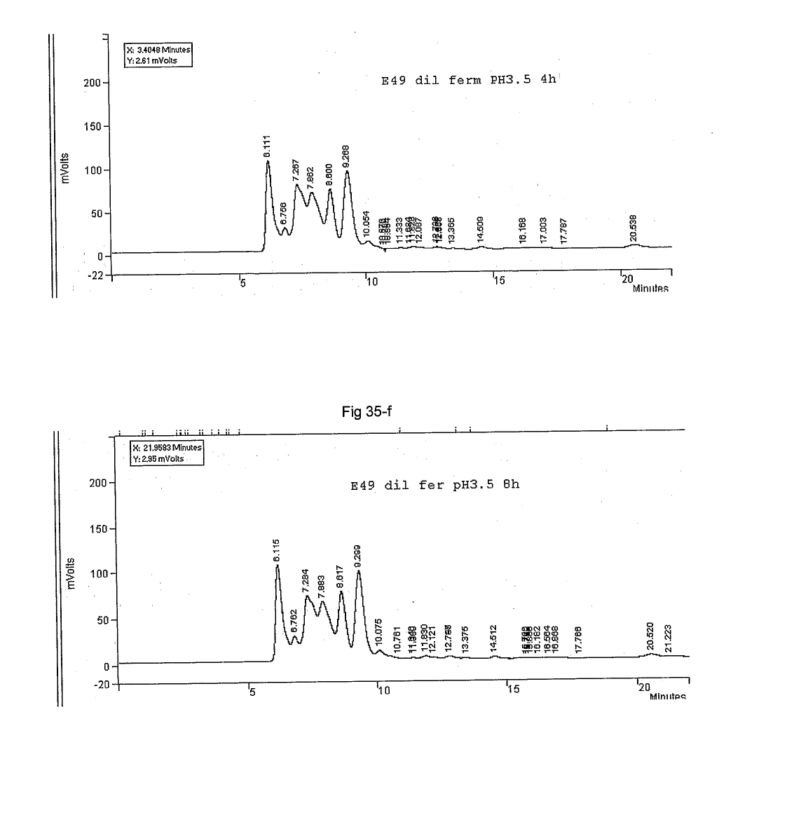

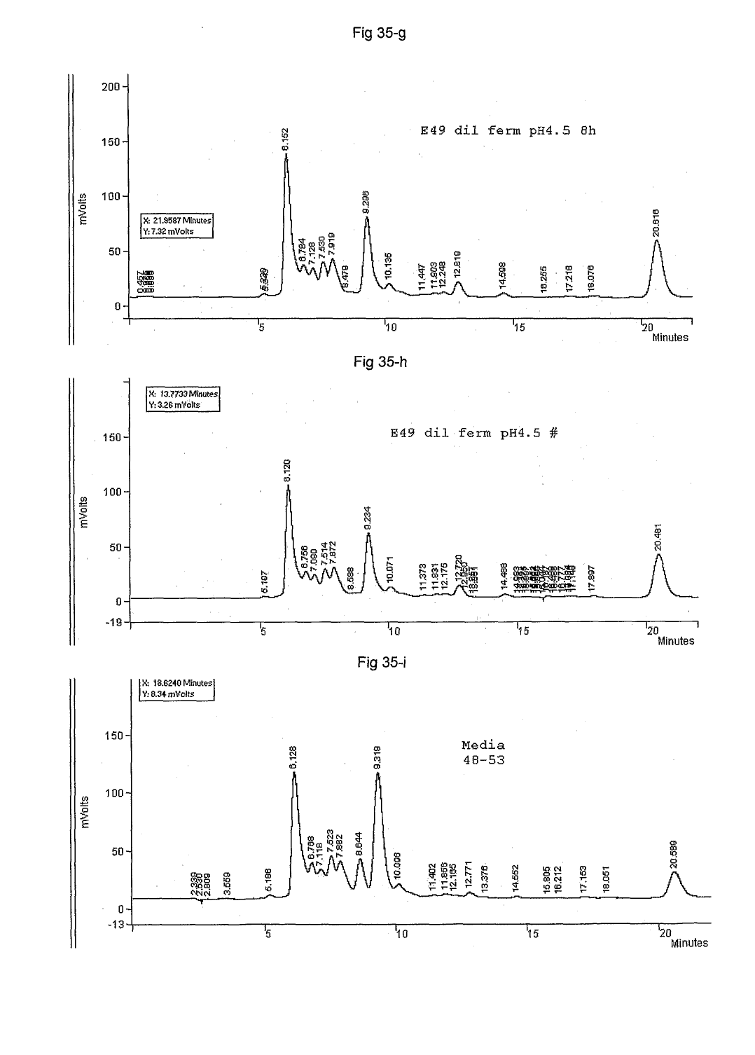

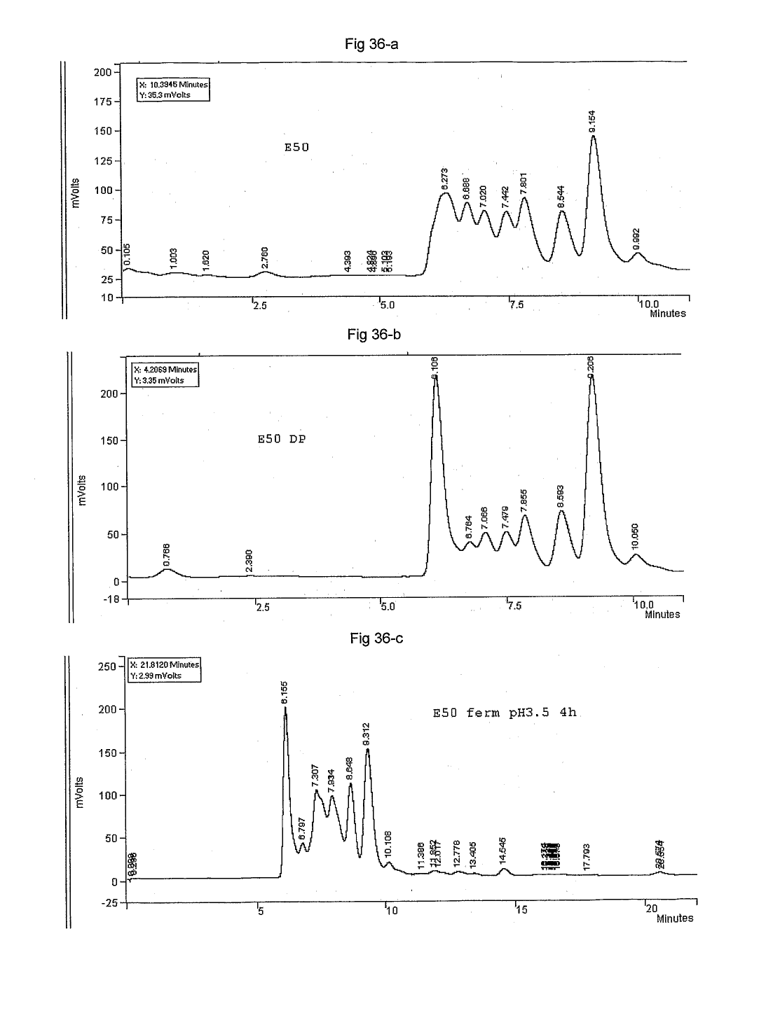

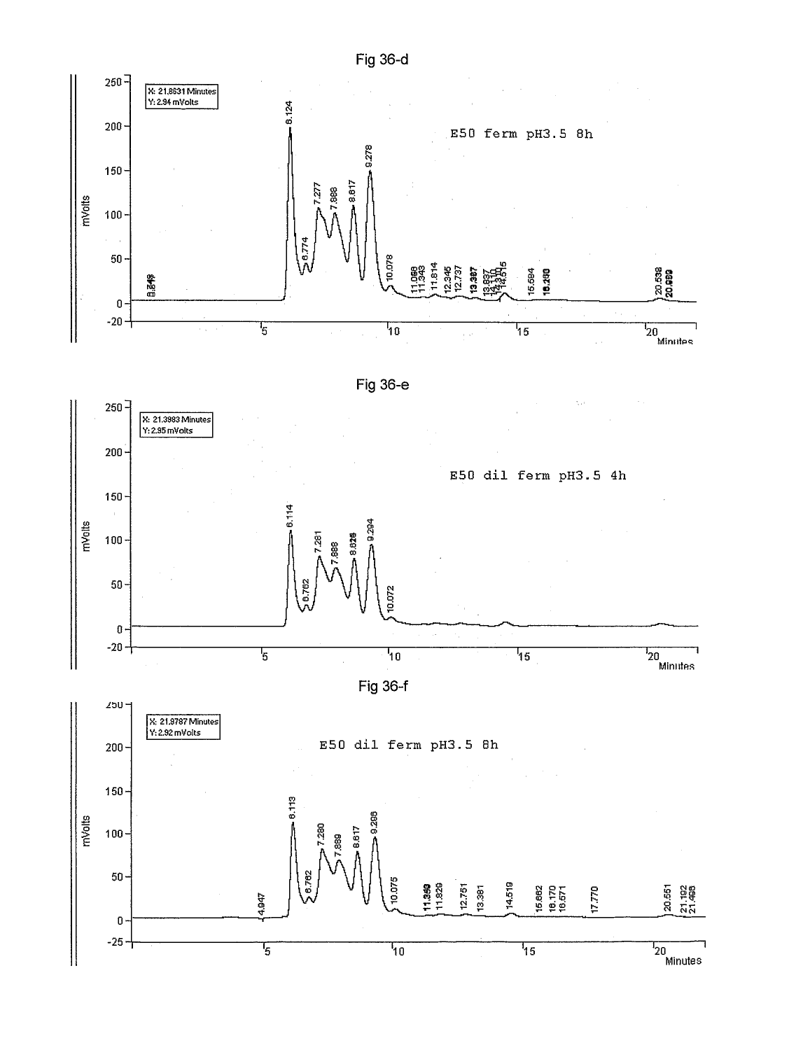

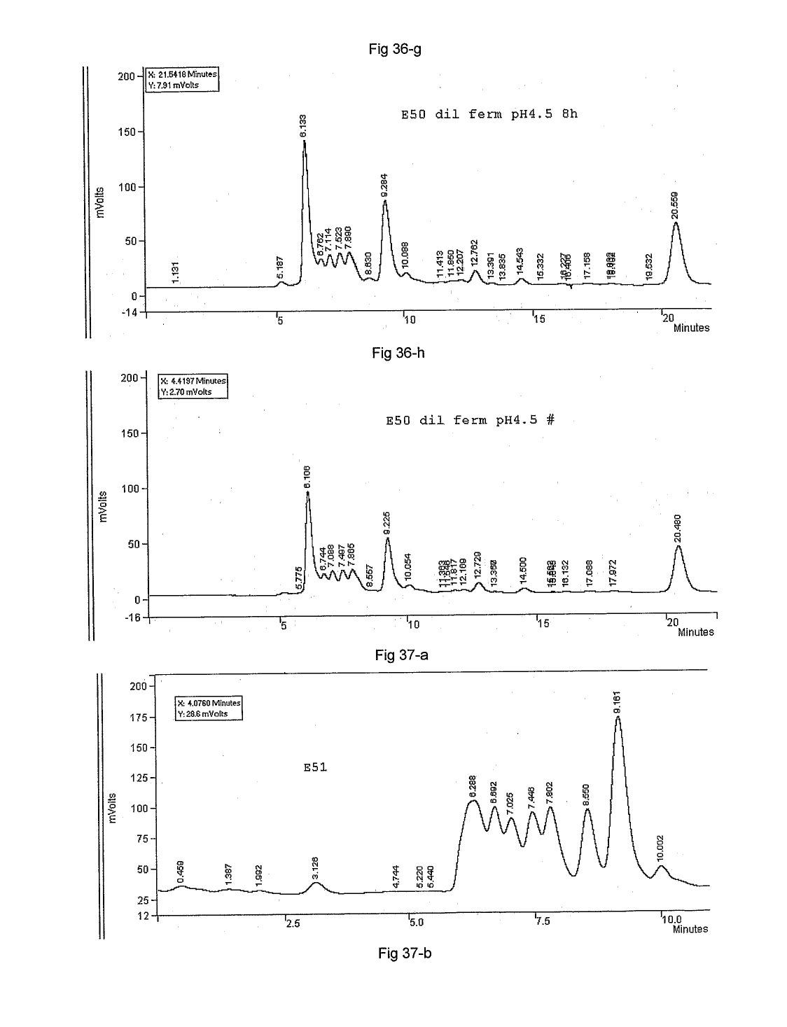

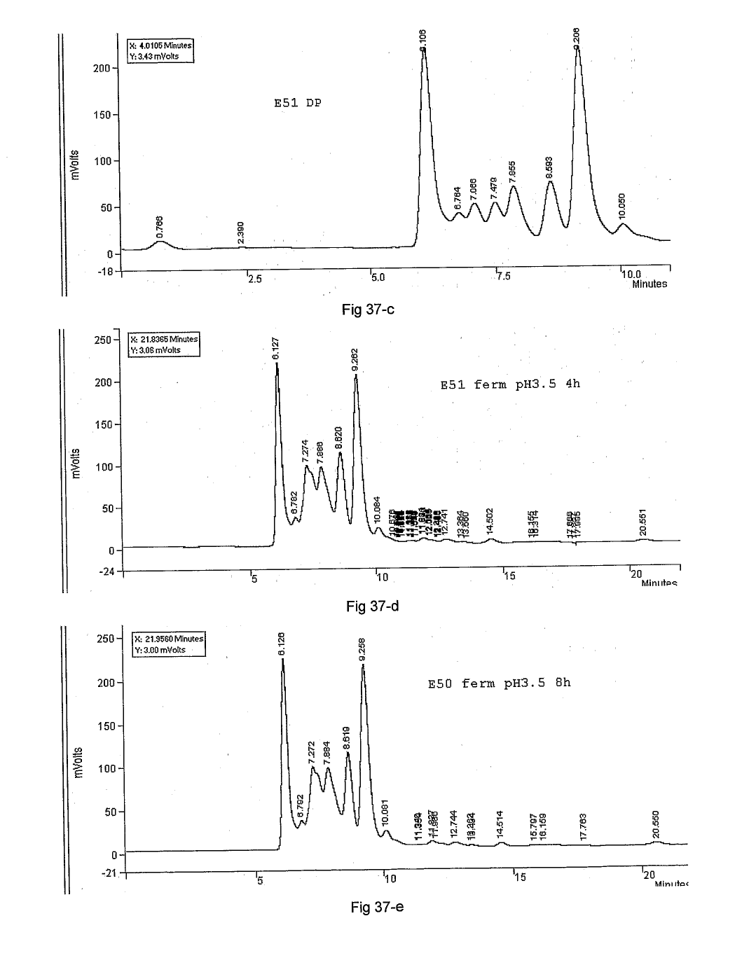

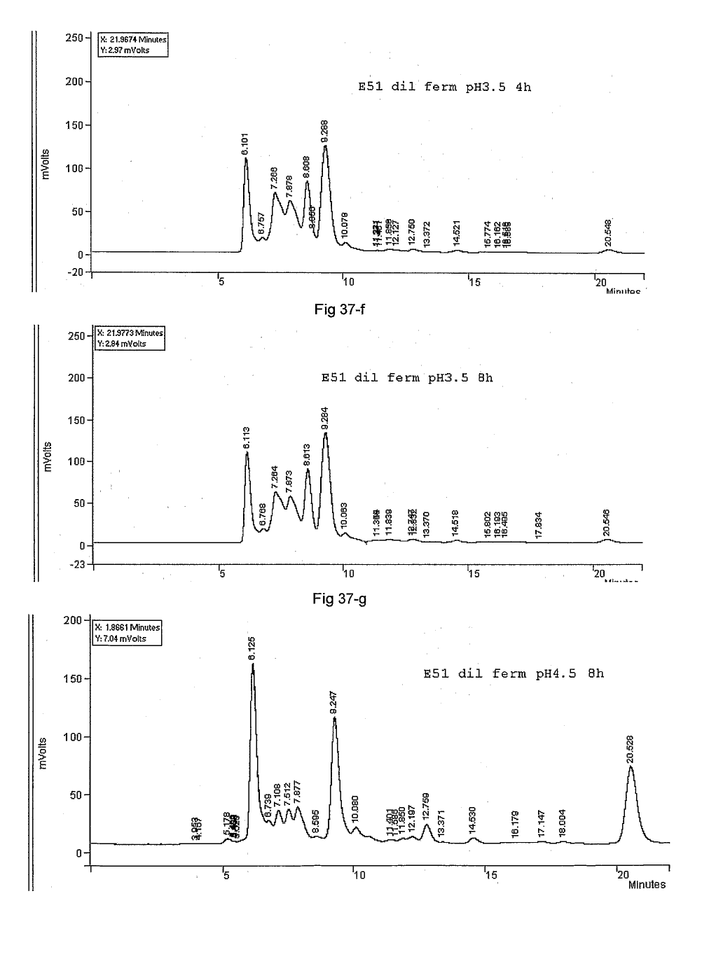

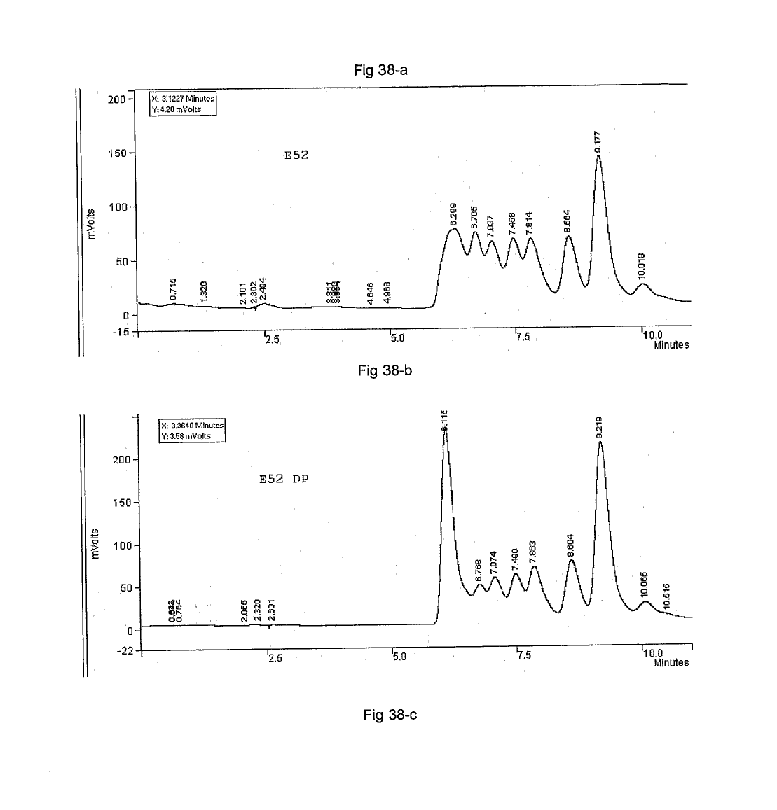

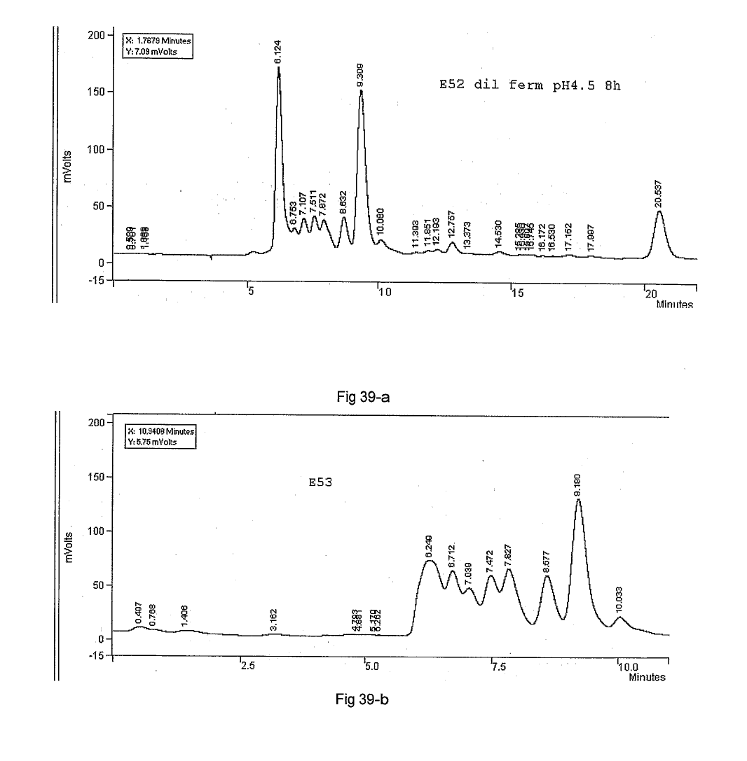

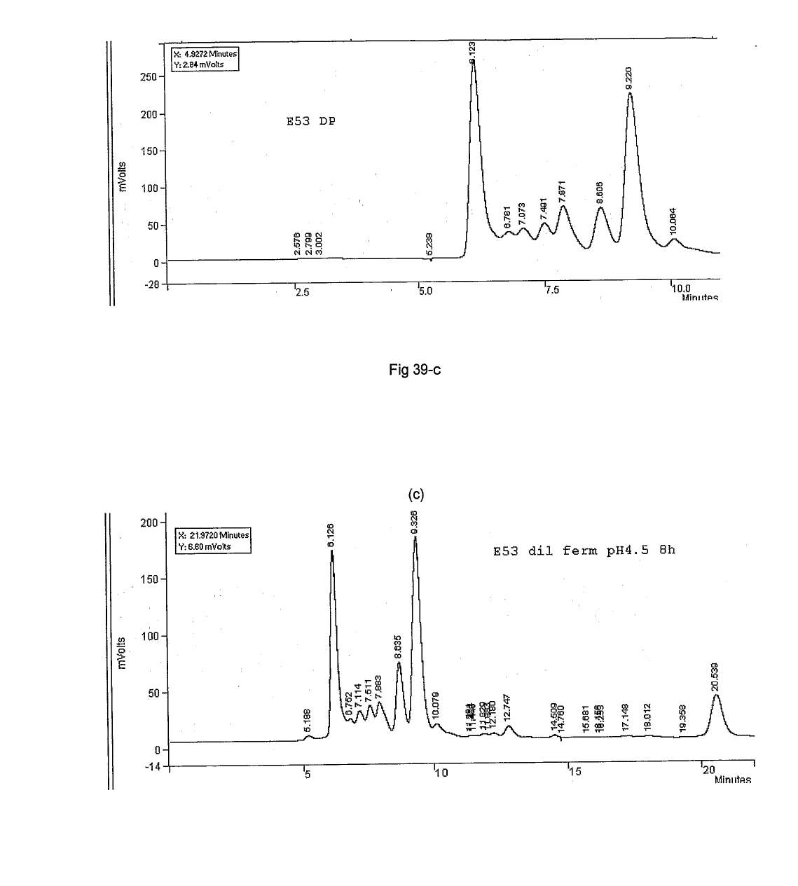

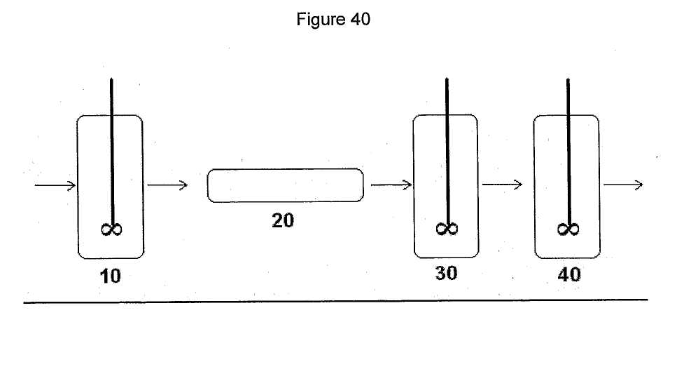

Immediately after the production of inoculum in the shaker, the subsequent preparations are performed in reactors designed for industrial production per se. Various types of reactors can be used for the purpose in the present invention, the preferred use being a vertical cylindrical reactor having chicanes to increase the transfer of mass and media for heat exchange, for when it is necessary to adjust the temperature of the reactor. An example merely for illustration purposes is shown in FIG. 40, reactor 10.

### EXAMPLE 3

### Sugar Cane Bagasse Pretreatment Method

The pretreatment method and enzymatic hydrolysis of sugar cane bagasse, mainly viewing the production of ethanol, described in the present invention is innovative and is designed to fill in gaps found in the state of the art. The technology described herein differs from existing technologies, and is highly efficient in the process of converting the carbohydrates present in sugar cane bagasse into monosaccharides and, consequently, provides greater and more economically feasible production methods of ethanol (bioethanol) from sugar cane bagasse.

In a preferred embodiment of the invention, alkaline methods were used to perform the pretreatment of sugar cane bagasse and enzymes of Penicillium echinulatum for the enzymatic hydrolysis of the pretreated biomass. In this preferred embodiment, the fungus used is a filamentous fungus developed over the last 10 years at the University of Caxias do Sul by successive mutageneses and selections, having been identified as a good producer of cellulases and xylanases and deposited at the German resource center for biomaterial Deutsche Sammlung von Mikroorganismen und Zellkulturen (DSM) under the number DSM18942, strain 9A02S1. Excellent conversions of the complex carbohydrates of sugar can bagasse pretreated in reductive sugars were obtained.

The method of a preferred embodiment of the invention is now described in greater detail. Initially the sugar cane bagasse is dry and ground. The smaller the particles of lignocellulosic residue, the better the performance of pretreatment and the enzymatic hydrolysis. Fragments of up to 1 cm should be used. Next, a solution of sodium hydroxide is added whereby to obtain concentrations within the range of 1 to 4% (m/m) in relation to the lignocellulosic residue mass. Otherwise, hydrogen peroxide and/or anthraquinone can be used in concentrations within the range of 0.1 and 2% and 0.001 to 2%, respectively. This mixture is kept at temperatures in the range of 20 to 150° C., with the presence or absence of pressure, for between 30 minutes and up to 5 days. Duration times of over 5 days can be used in the pretreatments carried out at ambient temperature, without prejudicing the method. Afterwards, the pH is adjusted to within the range of 4 to 7 with sulfuric acid. This pretreated biomass is hydrolyzed using quantities of enzymes in the range of 10 to 15 FPU, in addition to quantities over 5 U of endoglycanases, 3 U of β-glycosidases, 2 U of xylanases per gram of dry biomass, keeping the reactional medium at temperatures in the range of 40 to 60° C., for 24 to 48 hours. Greater hydrolysis times do not interfere with the result, but should be avoided, because microbial contaminations may occur. The fermentable sugars obtained are fermented, with a view to producing ethanol and/or other products. It is known in literature that the glucose and xylose obtained in hydrolysis are converted into ethanol by yeasts.

### EXAMPLE 4

### Method for the Obtainment of Hydrolytic Enzymes—Pretreatment of Sugar Cane Bagasse with Steam

In the present preferred embodiment, the method of producing enzymes is preceded by a pretreatment of the bagasse to increase the degree of access of the enzymes to the substrate. Said pretreatment can be performed using various forms known in the state of the art, and preferably in this present invention the steam treatment method is used with or without the addition of homogeneous and/or heterogeneous catalyzers. Said pretreatment method is conducted under the following conditions: temperature between 100 and 200° C., preferably approximately 207° C., pressure of 10 to 25 bar, preferably 17 bar and residence time of 5 to 20 minutes, preferably 7 min., using sugar cane bagasse with a humidity level between 10 and 80%. Soon after the indicated temperature and time have been reached, a valve should be opened to allow a sudden or gradual decompression of the content, causing the release of part of the hemicelluloses present in the biomass. Table 6 shows the compositions of the bagasse before and after steam pretreatment:

TABLE 6
composition of the bagasse before and after steam pretreatment

| | Pretreated | | Bagasse | |
|---|---|---|---|---|
| | bagasse | | “in natura” | |
| | Original | | Original | |
| Determination | matter | Dry matter | matter | Dry matter |
| Dry matter (MS) | 40.78 | 100 | 54.56 | 100 |
| Gross Protein (PB) | 1.12 | 2.75 | 1.12 | 2.05 |
| Gross Fiber (FB) | 17 | 41.69 | 29.98 | 49.46 |
| Ether Extract (EE) | 0.24 | 0.6 | 0 | 0 |
| Mineral material | 0.95 | 2.33 | 2.08 | 3.82 |
| Non-nitrogenated extract | 21.46 | 52.63 | 24.37 | 44.88 |
| NDT (estimate according | 20.45 | 50.16 | 24.38 | 44.87 |
| to Kearl, L.C 1982) | | | | |
| Fiber in neutral detergent | 24.18 | 59.3 | 45.21 | 82.87 |
| Fiber in acid detergent | 22.21 | 54.46 | 34.71 | 63.63 |
| Insoluble ash | 0.17 | 0.43 | 1.22 | 2.24 |
| Lignin in acid detergent | | | | |
| Hemicelluloses(*) (8-9) | | | | (8-9) |
| Celluloses (9-(10-11) | | | | (9-(10-11) |
| True digestion “In vitro” | 24.4 | 59.83 | 24.6 | 45.1 |
| N of FDA | | | | |
| Ammoniacal-N | | | | |
| N in original matter | | | | |
| Protein digestible in pepsin | | | | |
| Non-structured carbohydrates | 14.46 | 35.45 | 1.37 | 13.51 |
| | | | | pH = 5.7 |

(*)calculated

Numerous experiments were conducted to evaluate the performance of the methods the invention, as illustrated in Table 7.

TABLE 7
Matrix of Experiments relating to the Enzymatic Hydrolysis of
Pretreated bagasse with Steam

| | Bagasse | | Enzyme | | Citrate | | Tween | Hydrolysis | | |
|---|---|---|---|---|---|---|---|---|---|---|
| | | Quant. | | Quant. | buffer | Water | 80 | Hydro | Time | Mechanical |
| | Type | (g) | Type | (g) | (g) | (g) | (g) | Module | (h) | Action |
| 1 | NT | 19.5 | RT3-96 | 120 | 100 | 10.40 | 0.25 | 12.83 | 22 | YES |
| 2 | NT | 19.5 | RT3-96 | 240 | 130 | 10.10 | 0.40 | 20.51 | 22 | YES |
| 3 | NT | 26.0 | RT3-96 | 120 | 40 | 13.75 | 0.25 | 7.69 | 22 | YES |
| 4 | NT | 52.0 | RT3-96 | 280 | 40 | 27.60 | 0.40 | 7.69 | 22 | YES |
| 5 | NT | 39.0 | RT3-96 | 350 | 80 | 340.60 | 0.40 | 20.77 | 22 | YES |
| 11 | TgL | 60.0 | RT6-60 | 120 | 60 | 510.00 | 0.03 | 11.50 | 9 | YES |
| 12 | TgL | 60.0 | RT6-60 | 120 | 60 | 510.00 | 0.03 | 11.50 | 22 | PARTIAL |
| 13 | TgL | 60.0 | RT8-71 | 30 | 60 | 510.00 | 0.03 | 11.00 | 9 | YES |
| 14 | Tg | 60.0 | RT8-71 | 60 | 60 | 480.00 | 0.03 | 11.00 | 8.5 | YES |
| 15 | TgL | 60.0 | RT8-71 | 60 | 60 | 480.00 | 0.03 | 11.00 | 12 | YES |
| 16 | TgL | 60.0 | RT8-71 | 60 | 60 | 480.00 | 0.03 | 11.00 | 22 | PARTIAL |
| 17 | TgL | 60.0 | RT8-71 | 120 | 60 | 320.00 | 0.03 | 11.00 | 14 | PARTIAL |
| 18 | TgL | 60.0 | RT8-71 | 60 | 60 | 480.00 | 0.03 | 11.00 | 10 | YES |
| 19 | TfL | 60.0 | RT8-71 | 120 | 60 | 480.00 | 0.03 | 11.73 | 24 | NO |
| 24 | TfL | 60.0 | RT8-71 | 120 | 60 | 460.00 | 0.03 | 11.67 | 12 | NO |
| 26 | TfL | 60.0 | RT8-71 | 120 | 60 | 390.00 | 0.03 | 10.50 | 20 | NO |
| 28 | TfL | 60.0 | RT8-71 | 120 | 60 | 390.00 | 0.03 | 10.50 | 20 | NO |
| 30 | Tf | 64.0 | RT8-71 | 120 | 60 | 411.00 | 0.03 | 10.29 | 6 | YES |
| 32 | Tf | 90.0 | RT8-71 | 160 | 80 | 630.00 | 0.03 | 10.67 | 6 | YES |
| 33 | Tf | 90.0 | RT-13 | 160 | 80 | 630.00 | 0.03 | 10.67 | 6 | YES |
| 34 | Tf | 90.0 | RT-13 | 160 | 80 | 630.00 | 0.03 | 10.67 | 6 | YES |
| 35 | Tf | 90.0 | RT-13 | 160 | 80 | 630.00 | 0.03 | 10.67 | 6 | YES |
| 36 | Tf | 90.0 | RT-13 | 160 | 80 | 630.00 | 0.03 | 10.67 | 6 | YES |
| 38 | Tf | 90.0 | Celluclast | 18.5 | 80 | 772.00 | 0.03 | 10.67 | 4 | YES |
| 39 | Tf | 90.0 | RT-13 | 160 | 80 | 630.00 | 0.03 | 10.67 | 4 | YES |
| 40 | Tf | 90.0 | RT8-71 | 160 | 80 | 630.00 | 0.03 | 10.67 | 12 | YES |
| 42 | Tf | 80.0 | RT8-71 | 160 | 80 | 640.00 | 0.03 | 12.00 | 9 | YES |
| 44 | TfL | 90.0 | RT8-71 | 160 | 80 | 630.00 | 0.03 | 10.67 | 12 | YES |
| 45 | TfL | 90.0 | RT8-71 | 160 | 80 | 630.00 | 0.03 | 10.67 | 9 | YES |
| 46 | Tf | 78.0 | RT8-71 | 160 | 80 | 630.00 | 0.03 | 12.50 | 12 | YES |
| 48 | Tf | 117.0 | RT8-71 | 160 | 80 | 797.00 | 0.03 | 9.86 | 12 | YES |
| 49 | Tf | 108.0 | RT8-71 | 140 | 100 | 866.00 | 0.03 | 11.24 | 12 | YES |
| 50 | Tf | 55.0 | RT8-71 | 110 | 80 | 415.00 | 0.03 | 12.00 | 12 | YES |
| 51 | Tf | 60.0 | RT8-71 | 120 | 80 | 460.00 | 0.03 | 12.00 | 12 | YES |
| 52 | Tf | 55.0 | RT8-71 | 120 | 80 | 382.00 | 0.03 | 11.57 | 12 | YES |
| 53 | Tf | 60.0 | RT8-71 | 120 | 80 | 475.00 | 0.03 | 12.25 | 12 | YES |

NB: Experiments # 12, # 16 and # 17 under mechanical action for 9 hours, 3 hours and 6 hours, respectively. Experiment # 50 used bagasse with 40% humidity. Experiments # 51 and # 53 used bagasse with 30% humidity.
NT: not crushed.
Tg: crushed in thresher g.
Tm: crushed in thresher m.
Tf: crushed in thresher f.
TgL: crushed in thresher g and then washed.
TfL: crushed in thresher f and then washed.
RT N-J: Enzyme produced in batch N for J hours.

### EXAMPLE 5

### Hydrolytic Method for Producing Fermentable Sugars

Enzymatic hydrolysis is characterized by the production of carbohydrates in the form of monomers and oligomers arising from the action of the enzymes produced under the conditions defined above, on the sugar cane bagasse pretreated with steam under the conditions defined above.

Formulation of the reactional medium: for each kg of medium (final relative volume by weight of products)

Sol. Enzyme A*=60% by PB

Sol. Enzyme B*=140% by PB

PB=Weight of bagasse=8-9%=Humidity 30-40%

Surfactant=0.5 g

Corrective pH=200 microliters of Sodium Hydroxide 55% or Sulfuric Acid 98%, so that the pH is between 4.8 and 5.5.

Fill up with water for the final volume of 1 Liter.

In a preferred embodiment of the invention, we considered an initial percentage of 200% of enzyme solution, obtained under the conditions defined as per the optimized activities under the conditions described above. In feeding, an absolute volume of 60% with enzymes is preferred, but in the 140% what is really important are activities originating in the ultrafiltration system that should be at least 70% of the initial activities.

It is estimated that the enzymatic complex responsible for hydrolysis basically comprises exoglucanases, endoglucanases and xylanases. Table 8 presents the average activities of these enzymes in different experiments.

TABLE 8
enzymatic activity obtained in diverse experiments

| Order | Sample | FPA | Beta | Xylanase | Endo |
|---|---|---|---|---|---|
| 1 | Rt6-96 | 0.4026 | (0.0446) | 0.1513 | 4.7636 |
| 2 | Rt8-71 | 0.6729 | 0.0292 | 0.2410 | 3.0400 |
| 3 | Rt10-86 | 0.5934 | 0.0905 | 0.1010 | 2.4220 |
| 4 | Rt12-49 | 0.3511 | 0.1566 | 0.1131 | 1.4300 |
| 5 | Rt13-48 | 0.6528 | (0.0560) | 0.1707 | 5.0100 |
| 6 | Rt5-48 | 0.0510 | 0.0426 | 0.2551 | 6.32 |

Key:
Rtx-y = Experiment of no. x in fermentation time y.

In the activities described in Table 8, samples 1 to 5 were performed on exploded and washed bagasse, while in sample 6 the bagasse was not washed. This demonstrated that in condition 6 there is increased enzyme activity. This fact can be explained because in the explosion of the bagasse, the non-structured carbohydrates expose the hemicelluloses and celluloses, thus allowing greater substrate availability for induction in the production of enzymes, mainly xylanases and endoglucanases.

### Preparing the Hydrolysate

As a means of assuring that the bagasse continues to absorb the enzyme solution, in the entrance of the hydrolysis reactor, the preliminary mixture of these should preferably be 1 part pretreated bagasse pretreated to 2 parts enzyme and 2 parts water. Immediately after this procedure, the remainder of the water and the nutrients are added to the system, preferably on a continual basis. The hydrolysis time is preferably 12 hours, and this may be increased or not depending on the need to produce greater quantities of sugars. The method of enzymatic hydrolysis of bagasse of the present invention can be conducted using different equipment, preferably equipment that provides a high transfer of mass, which in general is provided by horizontal cylindrical equipment having chicanes and/or elements to increase the impact, as well as means for increasing the media for heat exchange, for when it is necessary to adjust the temperature of the reactor. An example merely to illustrate this is shown in FIG. 40, hydrolysis reactor 20.

### EXAMPLE 6

### Reuse of Enzymes in the Hydrolytic Method

### Preparation of Hydrolysate

Soon after the exit of the hydrolysate, the solution containing sugars (monomers and oligomers) and enzymes is filtered. At a first moment, the entire liquid is returned to the start of the hydrolysis method, with the purpose of incrementing the concentration of sugars. At a second moment, this solution undergoes a system of ultrafiltration, with threasher of 10 KD in quantities retained and permeated as determined by the desired concentration of sugars. The material retained in the ultrafiltration can be reused at the start of the following hydrolysis operation.

### EXAMPLE 7

### Hydrolytic Method—Chemical Hydrolysis

With the objective of transforming the oligomers into smaller sugars (monomers and dimers), of the hydrolysate available for the fermentation process, depolymerization or acid chemical post-hydrolysis is performed under a pressure of 1-2 bar and temperature of 120±20° C. for 20-60 min. The saccharidic solution obtained must be filtered and sent for fermentation. If there is a need to concentrate the saccharidic solution, this process may be carried out simultaneously with the depolymerization/post-hydrolysis stage per se, using the energy employed for this purpose. The method of chemical hydrolysis of bagasse of the present invention can be conducted using different equipment, preferably equipment that provides a high transfer of mass, which in general is provided by horizontal cylindrical equipment having chicanes and/or elements to increase the impact, as well as means for increasing the media for heat exchange, for when it is necessary to adjust the temperature of the reactor. An example merely for illustrative purposes is shown in FIG. 40, hydrolyzed reactor 30.

### Sugars:

As a result of acid chemical hydrolysis of the enzymatic hydrolyte, fermentable sugars are produced. FIGS. 1-39 shows the data relating to the different methods carried out by the inventors.

### EXAMPLE 8

### Additives Comprising Fermentable Sugars

The fermentable sugars produced by the hydrolytic methods of the invention are useful in various applications, notably as industrial processing additives in diverse sectors. Preferably, but not limitedly, the fermentable sugars of the invention constitute an excellent additive for the production of ethanol, when used in conventional fermentative methods to produce alcohol with Saccharomyces sp. FIGS. 1-39 show data relating to the use of additives of the invention in methods of producing ethanol.

### EXAMPLE 9

### Process for Producing Ethanol

Table 9 shows the increase in ethanol production of a conventional plant/distillery, obtained from using sugar cane bagasse as the raw material for producing ethanol. It is important to note that in cases where the production of ethylic alcohol is lower, there was a lower conversion of carbohydrate NI-01, is having a rate of polymerization≧2. In Example 11, when ethanolic production by fermentation was significantly higher, it can be perceived that the consumption and conversion of this carbohydrate were practically quantitative.

TABLE 9
Quantities of raw materials used and concentrations of sugars and ethanol formed in different stages

| | Bagasse | | Hydrolyte | | | | Treated hydrolyte | | | Fermentate | | | | Produc- | | |
|---|---|---|---|---|---|---|---|---|---|---|---|---|---|---|---|---|
| | | Pre- | | | | Xy- | Cell- | Glu- | Xy- | Cell- | | | | tion | Yield | |
| | Virgin | treated | L/ton | Cellob. | Glucose | lose | ob. | cose | lose | ob. | Glucose | Xylose | Ethanol | (L/ton | (g/g | Ethanol |
| | (g) | (g) | BPT | (g/L) | (g/L) | (g/L) | (g/L) | (g/L) | (g/L) | (g/L) | (g/L) | (g/L) | (g/L) | BPT) | GBV) | (L/TBV) |
| H1 | 91.8 | 78.0 | 12821 | | 1.76 | 0.78 | | | | | | | 14.19 | 218.78 | 0.15 | 185.96 |
| H2 | 56.5 | 48.0 | 20834 | | 2.52 | 1.05 | | | | | | | 6.53 | 163.60 | 0.11 | 139.06 |
| H11 | 105.9 | 90.0 | 11111 | 0.41 | 2.41 | 0.38 | 1.37 | 3.13 | 0.51 | | | | 19.2 | 256.55 | 0.17 | 218.06 |
| H12 | 105.9 | 90.0 | 11111 | 0.48 | 3.04 | 0.46 | 0.62 | 2.55 | 0.38 | | | | 12.48 | 166.75 | 0.11 | 141.74 |
| H48 | 110.1 | 93.6 | 10685 | 2.18 | 1.76 | 4.25 | 1.96 | 2.12 | 5.06 | 0.55 | <0.25 | 1.58 | 11.8 | 151.63 | 0.10 | 128.88 |
| H49 | 101.2 | 86.0 | 11628 | 1.28 | 1.58 | 3.48 | 1.07 | 1.16 | 3.80 | 0.43 | <0.25 | 1.56 | 12.81 | 179.12 | 0.12 | 152.25 |
| H50 | 98.0 | 83.3 | 12001 | 1.19 | 1.27 | 2.73 | 1.03 | 1.12 | 3.26 | 0.48 | <0.25 | 1.68 | 13.96 | 201.47 | 0.14 | 171.25 |
| H51 | 98.0 | 83.3 | 12005 | 1.28 | 1.53 | 3.28 | 1.01 | 1.09 | 4.87 | 0.47 | <0.25 | <0.25 | 16.3 | 235.31 | 0.16 | 200.01 |
| H52 | 98.0 | 83.3 | 12005 | 1.31 | 1.51 | 3.20 | 1.18 | 1.28 | 4.84 | 0.55 | 0.61 | 3.25 | 10.28 | 148.40 | 0.10 | 126.14 |
| H53 | 98.0 | 83.3 | 12005 | 0.96 | 1.31 | 2.95 | 0.85 | 0.92 | 4.99 | 0.41 | 1.36 | 4.01 | 9.05 | 130.65 | 0.09 | 111.05 |
| H31 | 105.9 | 90.0 | 11111 | 0.75 | 3.42 | 0.26 | 1.49 | 3.11 | 0.35 | | | | 2.98 | 39.82 | 0.03 | 33.85 |
| H40 | 105.9 | 90.0 | 11111 | 0.34 | 3.66 | 0.20 | 0.84 | 4.77 | 0.39 | 0.76 | 0.00 | <0.25 | 4.18 | 55.85 | 0.04 | 47.47 |
| H41 | 105.9 | 90.0 | 11111 | 0.63 | 3.76 | 0.37 | 1.14 | 3.73 | 0.50 | 0.75 | 0.00 | <0.25 | 3.74 | 49.97 | 0.03 | 42.48 |
| H42 | 105.9 | 90.0 | 11111 | 0.86 | 3.98 | 0.78 | 1.48 | 3.02 | 0.48 | 1.20 | 0.00 | <0.25 | 3.4 | 45.43 | 0.03 | 38.62 |
| H43 | 105.9 | 90.0 | 11111 | 0.51 | 2.09 | 0.26 | 1.03 | 3.34 | 0.43 | 0.82 | 0.00 | <0.25 | 3.53 | 47.17 | 0.03 | 40.09 |
| H44 | 105.9 | 90.0 | 11111 | 0.69 | 3.02 | 0.32 | 1.38 | 3.80 | 0.42 | 1.28 | 0.00 | <0.25 | 3.82 | 51.04 | 0.03 | 43.39 |
| H45 | 105.9 | 90.0 | 11111 | 0.54 | 2.48 | 0.27 | 1.57 | 3.03 | 0.37 | 1.55 | 1.66 | 0.91 | 4.49 | 59.99 | 0.04 | 51.00 |

BPT: Pretreated bagasse.
GBV: Glucose in Virgin Bagasse.
TBV: Ton Virgin Bagasse.

FIG. 40 shows a schematic representation of the methods of the present invention, which can be carried out sequentially or separately, and may optionally include recycles and/or complementary equipment at different points. In a preferred embodiment, the sequential operation of the methods of the invention provides production of 240 liters of ethanol per ton of sugar cane bagasse (or 33 liters of ethanol per ton of raw sugar cane). Persons skilled in the art will immediately recognize the value of the high productivity increase in ethanol provided by this present invention, since productivity of ethanol made from sugar cane, without using the bagasse provided by this present invention, to is approximately 75 liters of ethanol per ton of sugar cane. Consequently, the additional production of 33 liters of ethanol from bagasse represents approximately 44% in additional ethanol per ton of harvested sugar cane.

Persons skilled in the art will find the method now proposed to be valuable as an excellent alternative to similar existing methods. The methods of the present invention are the result of a combination of choice of microorganism, that is, the selection of strains by means of genetic improvement—using mutagenesis techniques and protoplast fusion; development/improvement of industrial culture methods; streamlined engineering of the methods of producing sugar cane bagasse carbohydrates by enzymatic hydrolysis, in addition to the ethanolic fermentation methods of the enzymatic hydrolytes produced from said biomass. These factors enable the development of reduced-cost methods with suitable productivity and yield levels. The examples described in the present invention should not be interpreted as limiting the spirit and scope of the present invention, defined in the claims appended hereto.

What is claimed is:

1. A method for the obtainment of hydrolytic enzymes comprising the submerged culture of fungus Penicillium echinulatum strain 9A02S1 (DSM18942) in industrial reactors, using sugar cane bagasse as substrate.

2. The method, according to claim 2, wherein said sugar cane bagasse is previously treated with steam.

3. The method, according to claim 2, wherein said sugar cane bagasse is submitted to temperatures in the range of 100-220° C., under pressure of 10-25 bar, for 5-20 minutes.

4. The method, according to claim 1, wherein said culture occurs at a temperature in the range of 28 to 31° C.

5. A hydrolytic method for producing fermentable sugars comprising the application of enzymes produced by fungus Penicillium echinulatum strain 9A02S1 (DSM18942), cultivated in industrial reactors, and using sugar cane bagasse as substrate.

6. The method, according to claim 5, wherein at least part of said enzymes are reused for subsequent hydrolysis cycles.

7. The method, according to claim 5, comprising a chemical hydrolysis stage in an industrial reactor.

8. An additive for industrial processing, comprising fermentable sugars generated from the enzymatic hydrolysis of sugar cane bagasse by enzymes produced by fungus Penicillium echinulatum strain 9A02S1 (DSM18942).

9. A method for producing ethanol, comprising the conversion into ethanol of fermentable sugars generated from the enzymatic hydrolysis of sugar cane bagasse by enzymes produced by the fungus Penicillium echinulatum strain 9A02S1 (DSM18942).

10. The method, according to claim 9, wherein said conversion is carried out by fermenting said fermentable sugars by Saccharomyces, in industrial reactors.

11. A method of pretreating sugar cane bagasse comprising at least one stage of alkaline treatment with sodium hydroxide, anthraquinone, hydrogen peroxide, or combinations thereof, with pH adjustment.

12. A culture media for industrial bioprocessing comprising sugar cane bagasse submitted to at least one stage of alkaline treatment with sodium hydroxide, anthraquinone, hydrogen peroxide, or combinations thereof, with pH adjustment.

13. A process for the industrial production of cellulases and/or hemicellulases comprising the culture of the fungus Penicillium echinulatum strain 9A02S1 (DSM18942).

14. The method, according to claim 6, comprising a chemical hydrolysis stage in an industrial reactor.
